(19) United States
(12) Patent Application Publication
(10) Pub. No.: US 2007/0254920 A1
(43) Pub. Date: Nov. 1, 2007

(54) Prodrug derivatives of acids using alcohols with homotopic hydroxy groups and methods for their preparation and use

(75) Inventors: Mitchell A. deLong, Raleigh, NC (US); Jill M. McFadden, Chapel Hill, NC (US); Susan M. Royalty, Cary, NC (US); Eric J. Toone, Durham, NC (US); Jeffrey D. Yingling, Apex, NC (US)

Correspondence Address: MICHAEL BEST & FRIEDRICH LLP, 100 E WISCONSIN AVENUE, Suite 3300, MILWAUKEE, WI, 53202, US

(73) Assignee: Aerie Pharmaceuticals, Inc., Research Triangle Park, NC (US)

(21) Appl. No.: 11/412,207

(22) Filed: Apr. 26, 2006

(51) Int. Cl. A61K 31/558; C07D 409/02; C07D 405/02; C07D 403/02

(52) U.S. Cl. 514/319; 514/323; 514/573; 514/415; 514/443; 514/469; 546/196; 546/201; 546/206; 548/495; 549/49; 549/462

(57) ABSTRACT

This invention relates to novel homotopic prodrugs and medicaments and methods for their preparation, testing and use. In one embodiment, the homotopic prodrug has the general formula 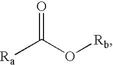
wherein 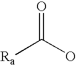
is a biologically-active moiety comprising a carboxylic acid functional group, and R_{b} is a homotopically-symmetrical alcohol bonded to the biologically-active moiety through the carboxylic acid functional group to form an ester linkage, as well as optical isomers, enantiomers, pharmaceutically acceptable salts, biohydrolyzable amides, esters, and imides thereof and combinations thereof.

### FIELD OF THE INVENTION

This invention relates to prodrug derivatives of carboxylic acids and methods for their preparation and use. More specifically, this invention relates to polyhydroxy alcohol esters possessing certain symmetry elements and methods for their preparation and use.

### BACKGROUND OF THE INVENTION

Many biologically-active materials have significant limitations in their use as drugs and medications due to physico-chemical properties that are incompatible with in vivo delivery via the desired dosing route. Other materials can not be used due to local side-effects that occur at the high concentrations of the dosed materials before they are properly dispersed in the body. Until recently, the solution to these problems was to go back to the laboratory and chemically-modify the core structure of the molecule to change its properties. Unfortunately, this often causes a loss of the desirable characteristics as well.

A relatively recent concept has emerged which holds some promise to solve both of these problems. The idea that a drug or medicament need not be dosed in active form, but in a form that requires modification by the organism before becoming active is similar to the solution that cells themselves use when making certain proteins. The cells create a preliminary protein, typically a hormone or enzyme, in an inactive form, and at the appropriate time and place, the active molecule is released by cleavage into its active form. The prefixes used by biologists to explain the relationship between an inactive precursor and an active hormone or enzyme are ‘pro’ and ‘pre-pro’ (e.g., pro-insulin) depending on if one release step is needed (pro) or two (pre-pro). Medicinal chemists have adopted this language to the idea that a ‘pro-drug’, or more commonly ‘prodrug’, is one that requires biological modification to effect the release of the active chemical. (See: Albert, A. “Chemical Aspects of Selective Toxicity”, Nature, 1958, 182:421-423; Roche, E. B. “Design of Biopharmaceutical Properties through Prodrugs and Analogs”, Washington, DC: American Pharmaceutical Association, 1977.)

While there are many enzymes in the body that are potentially capable of releasing a prodrug in its active form, there are relatively few that have been exploited to date. Phosphatases, amidases, and esterases have all seen application in the literature as activators of prodrugs. In addition, non-biological release mechanisms have been exploited, such as a chemical linkage that is sensitive to water, or low pH.

Prostaglandins and prostaglandin analogs are carboxylic acid derivatives that have often been preferentially dosed as prodrugs, particularly in ocular formulations. All naturally occurring prostaglandins have a carboxylic acid moiety at the C₁ position. The C₁ position is therefore the site for the chemical modification to create the prodrug moiety. Attempts have been made to modify the carboxylic acid moiety at the C₁ position as a sulfonamide moiety, and as a tetrazole (See: PCT Publication Nos. WO 99/12895, WO 99/12896, and WO 99/12898.) However, such modifications have either resulted in only modest increases in half-life or resulted in compounds with diminished potency. An alternative approach has been to replace C₁ with a heteroatom. For example, PGF analogs containing a sulfonic acid moiety at C₁ (see Iguchi, Y.; Kori, S.; Hayashi, M. “The chemistry of prostaglandins containing the sulfo group”, J. Org. Chem., 1975, 40, 521-523) and PGF analogs containing a phosphonic acid moiety at C₁ (see Kluender, H. C. & Woessner, W., Prostaglandins and Medicine, 1979, 2, 441-444) have been disclosed. However, such compounds suffer from significantly diminished potency.

Moreover, the Corey synthetic route to prostaglandins was specifically designed for a carboxylic acid moiety at C₁, and modifications are either incompatible with this efficient route or require significant optimization of difficult chemistry for each new C₁ replacement. Syntheses of prostaglandin analogs via the Corey route are described in the following references: Corey, E. J.; Weinshenker, N. M.; Schaaf, T. K.; Huber, W. J. Am. Chem. Soc., 1969, 91, 5675 and Corey, E. J.; Schaaf, T. K.; Huber, W.; Koelliker, U.; Weinshenker, N. M.; J. Am. Chem. Soc., 1970, 92, 397.

Thus, while a few prostaglandin analogs have been disclosed wherein C₁ has been replaced with a non acid moiety, there is a continuing need for suitable esters that can be used to modify the activity of the many potent, selective prostaglandin derivatives for the treatment of a variety of diseases and other conditions.

### SUMMARY OF THE INVENTION

In one embodiment, the invention provides a compound having the formula 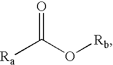
wherein 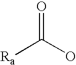
is a biologically-active moiety comprising a carboxylic acid functional group, and R_{b} is a homotopically-symmetrical alcohol bonded to the biologically-active moiety through the carboxylic acid functional group to form an ester linkage, as well as optical isomers, enantiomers, pharmaceutically acceptable salts, biohydrolyzable amides, esters, and imides thereof and combinations thereof.

In another embodiment, the invention provides a composition comprising A) a compound having the formula 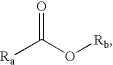
wherein 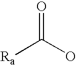
is a biologically-active moiety comprising a carboxylic acid functional group, and R_{b} is a homotopically-symmetrical alcohol bonded to the biologically-active moiety through the carboxylic acid functional group to form an ester linkage, as well as optical isomers, enantiomers, pharmaceutically acceptable salts, biohydrolyzable amides, esters, and imides thereof and combinations thereof.

In yet another embodiment, the invention provides method for treating a condition comprising administering to a subject in need of treatment, a homotopic prodrug of a prostaglandin EP₂ agonist, wherein the condition is selected from the group consisting of glaucoma, ocular hypertension, premenstrual tension, asthma and bone disorders.

In a further embodiment, the invention provides a method for treating a condition comprising administering to a subject in need of treatment, a homotopic prodrug of a prostaglandin EP₃ agonist, wherein the condition is selected from the group consisting of arthritis, bone disorders, vascular disease, hepatic diseases, renal diseases, pancreatitis, mycardial infarct, and gastric disturbances.

In another embodiment, the invention provides a method for controlling blood pressure comprising administering to a subject in need of treatment, a homotopic prodrug of an angiotensin-converting enzyme inhibitor.

In yet another embodiment, the invention provides a method for treating a condition comprising administering to a subject in need of treatment, a homotopic prodrug of a prostaglandin EP₄ agonist, wherein the condition is selected from the group consisting of glaucoma, neuroprotection, arthritis, bone disorders, vascular disease, and asthma.

In a further embodiment, the invention provides a method for treating a condition comprising administering to a subject in need of treatment, a homotopic prodrug of a prostaglandin FP agonist, wherein the condition is selected from the group consisting of glaucoma, skin disorders, circulatory disorders, gastrointestinal disorders, vascular diseases, and respiratory disorders.

In another embodiment, the invention provides a method for preventing premature labor comprising administering to a subject in need of treatment, a homotopic prodrug of a prostaglandin FP antagonist.

In yet another embodiment, the invention provides a method for treating sleeping disorders comprising administering to a subject in need of treatment, a homotopic prodrug of a prostaglandin DP agonist.

In a further embodiment, the invention provides a method for treating allergies comprising administering to a subject in need of treatment, a homotopic prodrug of a prostaglandin DP antagonist.

In another embodiment, the invention provides a method of ranking the susceptibility of an ester derivative to hydrolysis, said method comprising producing an ester derivative by reacting a biologically-active moiety having a carboxylic acid group with a homotopically-symmetrical alcohol that binds the biologically-active moiety through the carboxylic acid group to form an ester linkage, reacting the ester derivative with an enzyme that cleaves the ester linkage and releases the biologically-active moiety, measuring the rate at which the enzyme cleaves the ester linkage, and ranking the ester derivative for therapeutic usefulness.

### DETAILED DESCRIPTION OF THE INVENTION

This invention relates to novel hydroxy- or polyhydroxy-prodrug forms of drugs and medicaments including prostaglandins, ethacrynic acid derivatives and cephalosporins and methods for their preparation, testing, and use. These derivatives represent a significant advance over simple monofunctional hydrophobic esters as they are water-solubilizing, in contrast to the insoluble nature of the lower alkyl esters, and are thus particularly suitable for aqueous formulations. These derivatives also represent an advance over other attempts to create water-solubilizing prodrugs, as they are of well-defined structure, as opposed to the PEG, or polyethylene-glycol-type ester prodrugs, and are structurally a stable single isomer, as opposed to the sugar alcohol and glyceryl-type prodrugs.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

Publications and patents are referred to throughout this disclosure. All the U.S. Patents cited herein are hereby incorporated by reference.

All percentages, ratios, and proportions used herein are percent by weight unless otherwise specified.

### Definition and Usage of Terms

The following is a list of definitions for terms, as used herein:

“Acyl group” means a monovalent group suitable for acylation of a nitrogen atom to form an amide or carbamate or an alcohol to form an ester or a carbonate. Examples of acyl groups include, but are not limited to, benzoyl, acetyl, tert-butyl acetyl, para-phenyl benzoyl, and trifluoroacetyl, particularly, acetyl and benzoyl, and more particularly, acetyl.

“Agonist” means a compound that activates a receptor.

“Alcohol protecting group” means a group that replaces the active hydrogen of a hydroxyl moiety thus preventing undesired reactions at the hydroxyl moiety. Use of protecting groups in organic synthesis is well known in the art. Examples of protecting groups for alcohols may found in Chapter 2 Protecting Groups in Organic Synthesis by Greene, T. W. and Wuts, P. G. M., 2^{nd} ed., Wiley & Sons, Inc., 1991. Protecting groups include, but are not limited to, silyl ethers, alkoxymethyl ethers, tetrahydropyranyl ethers, tetrahydrofuranyl ethers, esters, and substituted or unsubstituted benzyl ethers.

“Antagonist” means a compound that inhibits a receptor.

“Aromatic group” means a monovalent group having a monocyclic ring structure or fused bicyclic ring structure. Monocyclic aromatic groups contain 5 to 10 carbon atoms, particularly 5 to 7 carbon atoms, and more particularly 5 to 6 carbon atoms in the ring. Bicyclic aromatic groups contain 8 to 12 carbon atoms, particularly 9 or 10 carbon atoms in the ring. Bicyclic aromatic groups include groups wherein only one ring is aromatic or where both rings are aromatic. Aromatic groups may be substituted or unsubstituted. One suitable aromatic group is phenyl.

“Biohydrolyzable” means capable of being hydrolyzed at a measurable rate in a biological system.

“Carbocyclic group” means a monovalent saturated or unsaturated hydrocarbon ring. Carbocyclic groups are monocyclic, or are fused, spiro, or bridged bicyclic ring systems. Monocyclic carbocyclic groups contain 3 to 10 carbon atoms, particularly 4 to 7 carbon atoms, and more particularly 5 to 6 carbon atoms in the ring. Bicyclic carbocyclic groups contain 8 to 12 carbon atoms, and more particularly 9 to 10 carbon atoms in the ring. Carbocyclic groups are unsubstituted. Examples of carbocyclic groups include, but are not limited to, cyclopentyl, cyclohexyl, cyclohexenyl, cycloheptyl, and cyclooctyl. The carbocyclic groups are particularly cyclopentyl, cyclohexyl, and cyclooctyl. In one embodiment, the carbocyclic group is cyclohexyl. Carbocyclic groups are not aromatic.

“Diol” means a moiety that contains two free hydroxyl groups.

“Free acid” means a carboxylic acid wherein the acidic proton has not been replaced with another moiety. Free acids are not protected and are not esters.

“Free hydroxyl” means a hydroxyl group wherein the acidic proton has not been replaced by another moiety. A hydroxyl group is also known as an alcohol, or —OH.

“Halogen atom” means F, Cl, Br, or I. In one embodiment, the halogen atom is F, Cl, or Br, more particularly Cl or F, and most particularly F.

“Halogenated hydrocarbon group” means a substituted monovalent hydrocarbon group or a substituted carbocyclic group, wherein at least one substituent is a halogen atom. Halogenated hydrocarbon groups can have a straight, branched, or cyclic structure. In some embodiments, halogenated hydrocarbon groups have 1 to 12 carbon atoms, more particularly 1 to 6 carbon atoms, and most particularly 1 to 3 carbon atoms. Suitable halogen atom substituents are Cl and F. One particularly suitable halogenated hydrocarbon group is trifluoromethyl.

“Heteroaromatic group” means an aromatic ring containing carbon and 1 to 4 heteroatoms in the ring. Heteroaromatic groups are monocyclic or fused bicyclic rings. Monocyclic heteroaromatic groups contain 5 to 10 member atoms (i.e., carbon and heteroatoms), particularly 5 to 7 member atoms, and more particularly 5 to 6 member atoms in the ring. Bicyclic heteroaromatic rings contain 8 to 12 member atoms and more particularly 9 or 10 member atoms in the ring. Heteroaromatic groups are unsubstituted. Examples of heteroaromatic groups include, but are not limited to, thienyl, thiazolyl, purinyl, pyrimidyl, pyridyl, and furanyl. In one embodiment, heteroaromatic groups include thienyl, furanyl, and pyridyl. One particularly suitable heteroaromatic group is thienyl.

“Heteroatom” means an atom other than carbon in the ring of a heterocyclic group or a heteroaromatic group or the chain of a heterogeneous group. Examples of heteroatoms include, but are not limited to, nitrogen, sulfur, and oxygen atoms. Groups containing more than one heteroatom may contain different heteroatoms.

“Heterocyclic group” means a saturated or unsaturated ring structure containing carbon and 1 to 4 heteroatoms in the ring. The attachment point for heterocyclic groups may be at one or more carbon atoms, one or more nitrogen atoms (if present) or a combination of carbon and nitrogen atoms. Heterocyclic groups are not aromatic. Heterocyclic groups are monocyclic, or are fused or bridged bicyclic ring systems. Monocyclic heterocyclic groups contain 3 to 10 member atoms (i.e., including both carbon atoms and at least 1 heteroatom), particularly 4 to 7 member atoms, and more particularly 5 to 6 member atoms in the ring. Bicyclic heterocyclic groups contain 8 to 12 member atoms, particularly, 9 or 10 member atoms in the ring. Heterocyclic groups are unsubstituted. Examples of heterocyclic groups include 1,3-dioxalane, 1,3-dioxane, piperzyl, morpholinyl, tetrahydrofuranyl, tetrahydropyranyl, and piperdyl.

“Heterogeneous group” means a saturated or unsaturated chain containing 1 to 18 member atoms, where the member atoms include carbon atoms and at least one heteroatom. The chain may contain 1 to 12 member atoms, more particularly 1 to 6 member atoms, and most particularly 1 to 4 member atoms. The chain may be straight or branched. Some branched heterogeneous groups have one or two branches, particularly one branch. Some heterogeneous groups are saturated. Unsaturated heterogeneous groups have one or more double bonds, one or more triple bonds, or both. Some unsaturated heterogeneous groups have one or two double bonds or one triple bond. In some embodiments, the unsaturated heterogeneous group has one double bond. Heterogeneous groups are unsubstituted.

“Homotopic prodrug” means a biologically-active carboxylic acid moiety esterified to a homotopically-symmetrical alcohol.

“Homotopically-symmetrical alcohol” means that all the free hydroxyls in the moiety are homotopic, that is chemically equivalent. Hotopically-symmetrical alcohols include triols and tetraols.

“Homotopic groups” means groups that are not distinguishable under any achiral conditions. In order to have homotopic groups the molecule must have a finite axis of rotation. The only molecules which can not have homotopic groups are those whose point groups are Cₗ, Cₛ, Cᵢ, Cᵥ. In general homotopic groups are related by the rotation axis. Homotopic groups will react the same in all chemical reactions and produce the same product.

“Hydroxy” or “Hydroxyl” means a chemical entity that comprises —OH. Alcohols contain hydroxy groups. Hydroxy groups may be free or protected.

“Monofunctional” means that a chemical entity has only one functional group.

“Monofunctional alcohol” means a chemical entity which contains only one hydroxy functional group. Methanol, ethanol, and isopropanol are all examples of monofunctional alcohols.

“Monovalent hydrocarbon group” means a chain of 1 to 18 carbon atoms, particularly 1 to 12 carbon atoms, more particularly 1 to 6 carbon atoms. “Lower monovalent hydrocarbon group” means a monovalent hydrocarbon group having 1 to 4 carbon atoms, particularly 1 to 3 carbon atoms, more particularly 1 to 2 carbon atoms. Lower monovalent hydrocarbon groups can include alkyl groups such as methyl and ethyl. Monovalent hydrocarbon groups may have a straight-chain or branched-chain structure. In one embodiment, the branched monovalent hydrocarbon groups have one or two branches, particularly one branch. Monovalent hydrocarbon groups may be saturated. Unsaturated monovalent hydrocarbon groups have one or more double bonds, one or more triple bonds, or combinations thereof. Some unsaturated monovalent hydrocarbon groups have one or two double bonds or one triple bond, more particularly unsaturated monovalent hydrocarbon groups have one double bond.

“PEG” means polyethylene glycol.

“Pharmaceutically acceptable” means suitable for use in a human or other mammal.

“Prostaglandin” means a fatty acid derivative which has a variety of potent biological activities of a hormonal or regulatory nature, or a synthetic version thereof.

“Polyol,” “Polyhydroxyl” or “polyhydroxy” means a compound containing at least two free hydroxyl groups.

“Selective” means having a binding or activation preference for a specific receptor over other receptors which can be quantitated based upon receptor binding or activation assays.

“Subject” means a living vertebrate animal such as a mammal (preferably human) in need of treatment.

“Substituted aromatic group” means an aromatic group wherein 1 to 4 of the hydrogen atoms bonded to carbon atoms in the ring have been replaced with other substituents. Some substituents may include, but are not limited to, halogen atoms, cyano groups, monovalent hydrocarbon groups, substituted monovalent hydrocarbon groups, heterogeneous groups, aromatic groups, substituted aromatic groups, or any combination thereof, particularly, halogen atoms, monovalent hydrocarbon groups, and substituted monovalent hydrocarbon groups. Substituted aromatic groups may include naphthyl. The substituents may be substituted at the ortho, meta, or para position on the ring, or any combination thereof, particularly ortho or meta, and more particularly, ortho.

“Substituted carbocyclic group” means a carbocyclic group wherein 1 to 4 hydrogen atoms bonded to carbon atoms in the ring have been replaced with other substituents. Substituents may include, but are not limited to, halogen atoms, cyano groups, monovalent hydrocarbon groups, monovalent heterogeneous groups, substituted monovalent hydrocarbon groups, aromatic groups, substituted aromatic groups, or any combination thereof, particularly, halogen atoms and substituted monovalent hydrocarbon groups. Carbocyclic group does not include aromatic rings.

“Substituted heteroaromatic group” means a heteroaromatic group wherein 1 to 4 hydrogen atoms bonded to carbon atoms in the ring have been replaced with other substituents. Suitable substituents include, but are not limited to, halogen atoms, cyano groups (—C≡N), monovalent hydrocarbon groups, substituted monovalent hydrocarbon groups, heterogeneous groups, substituted heterogeneous groups, phenyl groups, phenoxy groups, or any combination thereof. More particularly substituents include halogen atoms, halogenated hydrocarbon groups, monovalent hydrocarbon groups, and phenyl groups.

“Substituted heterocyclic group” means a heterocyclic group wherein 1 to 4 hydrogen atoms bonded to carbon atoms in the ring have been replaced with other substituents. Some substituents may include, but are not limited to, halogen atoms, cyano groups, monovalent hydrocarbon groups, substituted monovalent hydrocarbon groups, heterogeneous groups, substituted heterogeneous groups, halogenated hydrocarbon groups, phenyl groups, phenoxy groups, or any combination thereof, particularly, halogen atoms and halogenated hydrocarbon groups. Substituted heterocyclic groups are not aromatic.

“Substituted heterogeneous group” means a heterogeneous group, wherein 1 to 4 of the hydrogen atoms bonded to carbon atoms in the chain have been replaced with other substituents. Substituents include, but are not limited to, halogen atoms, hydroxy groups, alkoxy groups (e.g., methoxy, ethoxy, propoxy, butoxy, and pentoxy), aryloxy groups (e.g., phenoxy, chlorophenoxy, tolyloxy, methoxyphenoxy, benzyloxy, alkyloxycarbonylphenoxy, and acyloxyphenoxy), acyloxy groups (e.g., propionyloxy, benzoyloxy, and acetoxy), carbamoyloxy groups, carboxy groups, mercapto groups, alkylthio groups, acylthio groups, arylthio groups (e.g., phenylthio, chlorophenylthio, alkylphenylthio, alkoxyphenylthio, benzylthio, and alkyloxycarbonylphenylthio), aromatic groups (e.g., phenyl and tolyl), substituted aromatic groups (e.g., alkoxyphenyl, alkoxycarbonylphenyl, and halophenyl), heterocyclic groups, heteroaromatic groups, substituted heterocyclic groups, substituted heteroaromatic groups, and amino groups (e.g., amino, mono- and di-alkylamino having 1 to 3 carbon atoms, methylphenylamino, methylbenzylamino, alkanylamido groups of 1 to 3 carbon atoms, carbamamido, ureido, and guanidino).

“Substituted monovalent hydrocarbon group” means a monovalent hydrocarbon group wherein 1 to 4 of the hydrogen atoms bonded to carbon atoms in the chain have been replaced with other substituents. Substituents may include, but are not limited to, halogen atoms, halogenated hydrocarbon groups, alkyl groups (e.g., methyl, ethyl, propyl, and butyl), hydroxy groups, alkoxy groups (e.g., methoxy, ethoxy, propoxy, butoxy, and pentoxy), aryloxy groups (e.g., phenoxy, chlorophenoxy, tolyloxy, methoxyphenoxy, benzyloxy, alkyloxycarbonylphenoxy, and acyloxyphenoxy), acyloxy groups (e.g., propionyloxy, benzoyloxy, and acetoxy), carbamoyloxy groups, carboxy groups, mercapto groups, alkylthio groups, acylthio groups, arylthio groups (e.g., phenylthio, chlorophenylthio, alkylphenylthio, alkoxyphenylthio, benzylthio, and alkyloxycarbonylphenylthio), aryl groups (e.g., phenyl, tolyl, alkoxyphenyl, alkoxycarbonylphenyl, and halophenyl), heterocyclic groups, heteroaryl groups, and amino groups (e.g., amino, mono- and di-alkanyl-amino groups of 1 to 3 carbon atoms, methylphenylamino, methylbenzylamino, alkanylamido groups of 1 to 3 carbon atoms, carbamamido, ureido, and guanidino).

“Symmetrical alcohol groups” means that the alcohol groups on a molecule, when unattached to any ester, are all related to each other by ‘symmetry’, as demonstrated by a symmetrical transformation, such as rotation or reflection in a mirror plane, and are thus all equal, or ‘degenerate’. This is easily determined by observation by one skilled in the art, or by the use of a machine that can differentiate between degenerate and unique hydroxyl groups, such as a proton NMR or a Carbon-13 NMR spectrometer.

“Symmetry method” means a specific method of determining the symmetry of an organic molecule. The symmetry method is the most sophisticated but the quickest method used in the art and requires that one determine if mirror planes, rotation axes or inversion centers that interchange atoms exist in a molecule. Atoms that can be interchanged are chemically equivalent to each other.

“Symmetry Operators” or “Symmetry Transformations” means taking an organic molecule in 3-dimensional space and performing one of the following operations on it to determine if the molecule is symmetrical with respect to the transformation being used. Schoenflies Notation of symmetry operations: Rotation Axis, Cₙ: if an imaginary line (or axis) can be drawn through a molecule so that rotation by 360°/n gives a molecule indistinguishable from the original, that molecule is said to have a rotation axis, Cₙ, of order n. Molecules may contain more than one rotation axis, the highest is the principle axis. Reflection Plane (σ): a molecule has a plane of symmetry if an imaginary double-sided mirror reflects both halves of the molecule into one another so that the new molecule is indistinguishable from the original. In other words the mirror plane divides the molecule into two symmetric halves, each a reflection of the other. Molecules may contain more than one mirror plane. Mirror planes which contain the principle axis are σᵥ and those perpendicular to the principle axis are σₕ, “Diagonal planes”, σ_{d}, are vertical planes that bisect the angles between successive C₂ axes. Rotation-Reflection (Sₙ): a combination of the two previous symmetry elements is a distinct symmetry element called a rotation-reflection. This can be described as: Sₙ=Cₙ×Cσₕ=σₕ×Cₙ, the order of operations is immaterial. Center of Symmetry (or Inversion (I)): a molecule has a center of symmetry if there is a point within the molecule such that reflection of all atoms through that point gives a new molecule which is indistinguishable from the original. The center of symmetry must occur where all rotation axis and mirror planes meet.

“Tetraol” means an alcohol that has four hydroxyl groups. A symmetrical tetraol has four symmetrical hydroxyl groups. The symmetry of the groups makes all four hydroxyl groups homotopic and thus chemically-equivalent. A non-limiting example of a tetraol using this definition is 2,2-bis(hydroxy methyl)-propane-1,3-diol.

“Triol” means an alcohol that has three hydroxyl groups. In symmetrical alcohols the three are homotopically-symmetrical hydroxyl groups. The symmetry of the groups makes all three hydroxyl groups homotopic and thus chemically-equivalent. A non-limiting example of a triol using this definition is 2-(hydroxymethyl)-2-methylpropane-1,3-diol.

### Prodrug Derivatives Using Alcohols with Homotopic Hydroxy Groups

There are found in many biologically-active molecules a carboxylic acid functional group, which can be masked by chemical modification to an ester functional group. In most cases, this modification severely reduces or completely eliminates the biological activity found in the ‘free’ acid (called ‘free’ as it is ‘free’ of derivitization). Thus the ester modification produces a prodrug that remains biologically inactive until esterases present in the tissue of interest hydrolyze the ester and release the active free acid.

The prodrug derivatives of this invention suitably have the general formula: 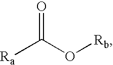
wherein 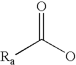
is a biologically-active moiety comprising a carboxylic acid functional group; and R_{b} is a homotopically-symmetrical alcohol bonded to the biologically-active moiety through the carboxylic acid functional group to form an ester linkage, as well as optical isomers, enantiomers, pharmaceutically acceptable salts, biohydrolyzable amides, esters, and imides thereof and combinations thereof.

Among the classes of drugs that contain a biologically-active moiety having a carboxylic acid functional group are the cephalosporin antibiotics, such as Cephalothin, Cephacetrile, Cephapirin, Cephaloridine, Cefazolin, Cefazuflur, Ceforanide, Cefazedone, Ceftezole, Cephanone, Cefotiam, Cefamandole, Cefonicid, Cefuroxime, Cefoperazone, Cefpiramide, Cefpimizole, Cefsulodin, Cefoxitin, Cefinetazole, Cefotetan, Cefbuperazone, Cefotaxime, Cefmenoxime, Ceftizoxime, Cefpirome, Ceftazidime, Cefodizime, Ceftriaxone, Latamoxef, Cephalexin, Cephradine, Cefaclor, Cefadroxil, Cefatrizine, Cefroxadine, and Cephaloglycin; the prostaglandins and prostaglandin derivatives, including the core structures of the ocular drugs tafluprost (AFP-168), Lumigan®, Travatan® and Xalatan®, the ethacrynic acids (and related compounds such as Ticrynafen and the various dihydrocinnamic acids and cinnamic acids such as such as SA9000 and SA8248 (Santen) (Shimazaki et al, Biol. Pharm. Bull. 27:1091-1024 (2004), Shimazaki et al, Biol. Pharm. Bull. 27:846-850 (2004)).

Further non-limiting examples of drugs that contain a biologically-active moiety comprising a carboxylic acid functional group and are specifically contemplated in this invention are: non-steroidal anti-inflammatory agents, such as Acetylsalicylic acid (aspirin), Salicylic acid, Sulindac, Indomethacin, Naproxen, Fenoprofen, Ibuprofen, Ketoprofen, Indoprofen, Furobufen, Diflunisal, Tolmetin, Flurbiprofen, Diclofenac, Mefenamic acid, Flufenamic acid, Meclofenamic acid, Fenclozic acid, Aldlofenac, Bucloxic acid, Suprofen, Fluprofen, Cinchophen, Pirprofen, Oxoprozin, Cinmetacin, Acemetacin, Ketorolac, Clometacin, Ibufenac, Tolfenamic acid, Fenclofenac, Prodolic acid, Clonixin, Flutiazin, Flufenisal, Salicylsalicylic acid, O-(Carbamoylphenoxy)acetic acid, Zomepirac, Nifluminic acid, Lonazolac, Fenbufen, Carprofen, Tiaprofenic acid, Loxoprofen, Etodolac, Alminoprofen, 2-(8-Methyl-10,11-dihydro-11-oxodibenz[b,f]oxepin-2-yl)-propionic acid, and 4-Biphenylacetic acid; Penicillin antibiotics, such as Benzylpenicillin, Phenoxymethylpenicillin, Phenethicillin, Methicillin, Nafcillin, Oxacillin, Cloxacillin, Dicloxacillin, Flucloxacillin, Azidocillin; Ampicillin, Amoxycillin, Epicillin, Cyclacillin, Carbenicillin, Ticarcillin, Sulbenicillin, Azlocillin, Mezlocillin, Piperazillin, Apalcillin, Temocillin, Carfecillin, Carindacillin, and Hetacillin; 4-Quinolone antibiotics, such as Ciprofloxacin, Norfloxacin, Acrosoxacin, Pipemidic acid, Nalidixic acid, Enoxacin, Ofloxacin, Oxolinic acid, Flumequine, Cinoxacin, Piromidic acid and Pefloxacin; angiotension-converting enzyme inhibitors, such as (2R,4R)-2-(2-Hydroxyphenyl)3-(3-mercaptopropionyl)-4-thiazolidinecarboxylic acid, Enalaprilic acid (N-[1-(S)-carboxy-3-phenyl-propyl]-L-alanyl-L-proline), Captopril, N-Cyclopentyl-N-[3-[(2,2-dimethyl-1-oxopropyl)thio]-2-methyl-1-oxopropyl]glycine, 1[4-Carboxy-2-methyl-2R,4R-pentanoyl]-2,3-dihydro-2S-indole-2-carboxylic acid, Alecapril (1-[(S)-3-Acetylthio-2-methyl-propanoyl]-L-propyl-L-phenylalanine), [3S-[2[R*(R*)]],3R*]-2-[2-[[1-carboxy-3-phenyl propyl]-amino]-1-oxopropyl]-1,2,3,4-tetrahydro-3-isoquinoline carboxylic acid, [2S-[1[R*(R*)]], 2α,3αβ, 7αβ,]-1[2-[[1-carboxy-3-phenylpropyl]-amino]-1-oxopropyl]octahydro-1H-indole-2-carboxylic acid, (S)-Benzamido-4-oxo-6-phenylhexanoyl-2-carboxy-pyrrolidine, Lisinopril, Tiopronin, Pivopril; and, other bio-affecting carboxylic acids, such as α-Methyl-L-tyrosine, Penicillamine, Probenicid, 5-Aminosalicylic acid, 4-Aminobenzoic acid, Methyldopa, L-Dopa, Carbidopa, Valproic acid, 4-Aminobutyric acid, Moxalactam, Clavulanic acid, Tranexamic acid, Furosemide, 7-Theophylline acetic acid, Clofibric acid, Thienamycin, N-Formimidoylthienamycin, Amphotericin B, Nicotinic acid, Methotrexate, L-Thyroxine, Cromoglycic acid, Bumetanide, Folic acid, Chlorambucil, Melphalan, Fusidic acid, 4-Aminosalicylic acid, Liothyronine, Tretinoin, o-Thymotinic acid, 6-Aminocaproic acid, L-Cysteine, Tranilast (N-(3′,4′-dimethoxycinnamoyl)anthranilic acid), Baclofen, 4-Amino-5-ethyl-3-thiophenecarboxylic acid, N-Cyclopentyl-N-[3-[(2,2-dimethyl-1-oxopropyl)thio]2-methyl-1-oxopropyl]glycine, Isoguvacine, Nipecotic acid, D-Eritadenine [(2R,3R)-4-adenin-9-yl-2,3-dihydroxybutanoic acid], (RS)-3-Adenin-9-yl-2-hydroxypropanoic acid, 1-[4-Carboxy-2-methyl-2R,4R-pentanoyl]-2,3-dihydro-2S-indole-2-carboxylic acid, Phenylalanylalanine, Glafenic acid, Floctafenic acid, N-(Phosphonoacetyl)-L-aspartic acid (PALA), Proxicromil, Cysteamine, N-Acetylcysteine, Proglumide, Aztreonam, Mecillinam, Retinoids and other nuclear hormone receptor ligands such as Bexarotene (Targretin®), All-trans-retinoic acid, 13-cis-retinoic acid, (Isotretinoin, CAS#4759-48-2, also known as Accutane®, Roaccutane® Amnesteem®, Isotane® and Sotret®), the various linolenic acids and the compounds disclosed in: US2004-0131648A1 and PCT Int. Appl. No. WO-04/03721 and incorporated herein by reference, Isonipecotic acid, Anthracene-9-carboxylic acid, α-Fluoromethylhistidine, 6-Amino-2-mercapto-5-methylpyrimidine-4-carboxylic acid, Glutathione, Acivicin, L-α-Glutamyl dopamine, 6-Aminonicotinic acid, Loflazepate, 6-[[1(S)-[3(S),4-dihydro-8-hydroxy-1-oxo-1H-2-benzopyran-3-yl]-3-methylbutyl]amino]-4-(S),5(S)-dihydroxy-6-oxo-3(S)-ammoniohexanoate, Z-2-Isovaleramidobut-2-enoic acid, D,L-2,4-Dihydroxyphenylalanine, L-2-Oxothiazolidine-4-carboxylic acid, lopanoic acid, 4-Aminomethylbenzoic acid, 4-Hydroxybenzoic acid, 4-Hydroxybutyric acid, 4-amino-3-phenylbutyric acid, 4-(Dimethylamino)benzoic acid, Capobenic acid, Pantothenic acid, Folinic acid, Orotic acid, Biotin, Mycophenolic acid, Thioctic acid, Pyroglutamic acid, Oleic acid, Linoleic acid, Cholic acid, Naturally occurring vitamins and amino acids (e.g. l-Tyrosine, glycine, histidine, niacin and glutamic acid), N,N-Dimethylglycine, Salazosulfapyridine, Azodisal, and Etretinic acid.

Prostaglandins and prostaglandin analogs are biologically-active moieties comprising a carboxylic acid functional group that have often been preferentially dosed as prodrugs, particularly in ocular formulations. In these cases, the isopropyl ester prodrug has long been the preferred form of ester prodrug. (See: Kerstetter, J. R.; Brubaker, R. F.; Wilson, S. E.; Kullerstrand, L. J. “Prostaglandin F₂ alpha-1-isopropylester lowers intraocular pressure without decreasing aqueous humor flow”, Am. J. Ophth., 1988, 105, 30-34). Naturally-occurring prostaglandins include PGE₁ and PGE₂, PGF_{2α}, prostacyclin (PGI), thromboxane and PGD₂. Naturally occurring prostaglandins have substituent groups at the C₉ and C₁₁ positions on the cyclopentyl ring, an optional cis double bond between C₅ and C₆, and a trans double bond between C₁₃ and C₁₄. Thus, the naturally occurring prostaglandins are exemplified by the following structures.

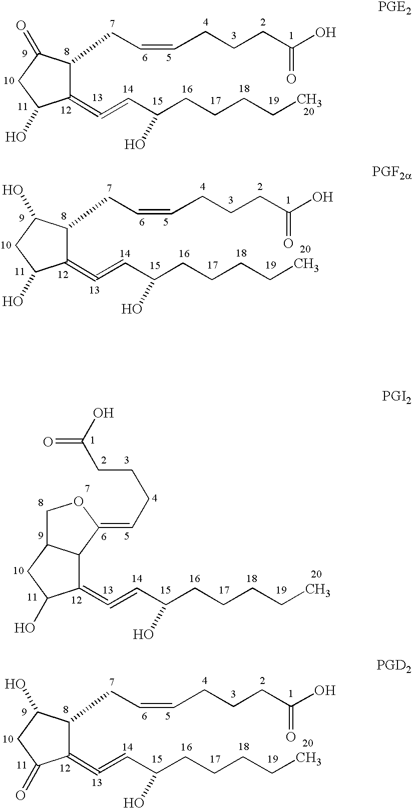

All naturally occurring prostaglandins have a carboxylic acid moiety at the C₁ position. The C₁ position is therefore the site for the chemical modification to create the prodrug moiety. Non-limiting examples of specifically-contemplated prostaglandins include: tafluprost (AFP-168), latanoprost free acid, unoprostone, fluprostenol, cloprostenol (both enantiomers of fluprostenol and cloprostenol as well as the racemate and all mixtures thereof are contemplated), 7-[3,5-dihydroxy-2-(3-hydroxy-5-phenyl-pent-1-enyl)-cyclopentyl]-N-ethyl-hept-5-enoic acid, tiaprost, Prostaglandin E₂ (PGE₂), butaprost, Prostaglandin F_{2α}(PGF_{2α}); 15-Deoxy-16-hydroxy-16-vinylprostaglandin E₂; 11-Deoxy-11_{α}, 12_{α}-methanoprostaglandin E₂; 11-Deoxy-11_{α},12_{α}-difluoromethano prostaglandin E₂; Prostacyclin; Epoprostenol; dl-16-Deoxy-16-hydroxy-16(α/β)-vinyl prostaglandin E₂; Prostaglandin E₁; Thromboxane A₂; 16,16-Dimethylprostaglandin E₂; (15R) 15-Methylprostaglandin E₂ (Arbaprostil); Meteneprost; Nileprost; and Ciprostene. Additional examples of prostaglandins can be found in U.S. Pat. No. 5,977,173 issued Nov. 2, 1999, U.S. Pat. No. 6,107,338 issued Aug. 22, 2000, U.S. Pat. No. 6,048,895 issued Apr. 11, 2000, U.S. Pat. No. 6,410,780 issued Jun. 25, 2002, U.S. Pat. No. 6,444,840 issued Sep. 3, 2002, U.S. Pat. No. 6,451,859 issued Sep. 17, 2002, U.S. Pat. No. 6,586,463 issued Jul. 1, 2003, U.S. patent application Ser. No. 09/774,555 filed Jan. 31, 2001, U.S. patent application Ser. No. 09/774,556 filed Jan. 31, 2001 and U.S. patent application Ser. No. 11/138097 filed May 26, 2005, which are hereby fully incorporated by reference.

Prostaglandin analogs have potent biological activities of a hormonal or regulatory nature. Examples of the biological activities and the conditions they can be used to treat are discussed below.

### PGE Analogs

Analogs of PGE are useful for treating a variety of medical conditions including pain. For example, EP₁ receptor antagonists have been used to block the pain induced by PGE₂ (see Ruel, R., Lacombe, P., Abramovitz, M., Godbout, C., Lamontagne, S., Rochette, C., Sawyer, N., Stocco, R., Tremblay, N. M., Metters, K. M., Labelle, M. “New class of biphenylene dibenzazocinones as potent ligands for the human EP₁ prostanoid receptor”, Bioorg. Med. Chem. Lett., 1999, 9, 2699-2704; and Hallinan, E. A., Hagen, T. J., Tsymbalov, S., Husa, R. K., Lee, A. C., Staplefield, A., Savage, M. A. “Aminoacetyl moiety as a potential surrogate for diacylhydrazine group of SC-51089, a potent PGE₂ antagonist, and its analogs”, J. Med. Chem., 1996, 39, 609).

Another use for PGE analogs is the treatment of arthritis, see Kiriyama, M., Ushikubi, F., Kobayashi, T., Hirata, M., Sugimoto, Y., Narumiya, S. “Binding specificities of the prostanoid receptors”, Br. J. Pharmacol., 1997, 122, 217-224; and Narumiya, S. “Roles of prostanoids in health and disease, lessons from receptor-knockout mice”, Int. Congr. Ser., 1999, 1181, 261-269.)

PGE analogs are also useful to treat bone disorders such as osteoporosis. Systemically administered PGE₂ was found to stimulate bone formation in dogs (see Shih, M. S., Norridin, R. W. “PGE₂ induces regional remodeling changes in Haversian envelope: A histomorphometric study of fractured ribs in beagles”, Bone and Mineral, 1986, 1, 227), in estrogen-depleted rats (see Mori, S., Jee, W. S. S., Li, X. J., Chan, S., Kimmel, D. B. “Effects of prostaglandin E2 on production of new cancellous bone in the axial skeleton of ovariectomized rats”, Bone, 1990, 11, 103) and to stimulate remodeling in the proximal tibia of estrogen-depleted rats (see Chyun, Y. S., Raisz, L. G. “Stimulation of bone formation by prostaglandin E2”, Prostaglandins, 1984, 27, 97). However, it is thought that more than one receptor may be responsible for the bone anabolic effects of PGE₂ (see Roof, S. L., deLong, M. A., Charest, R. P. “Messenger-RNA expression of prostaglandin receptors EP₁, EP₂, EP₃ and EP₄ in human osteoblast-like cells and 23 human tissues”, J. Bone Min. Res., 1996, 11, S337; Maruyama, T., Ohuchida, S. “New 3,7-dithiaprostanoic acid derivatives useful for the prevention and treatment of abnormal bone formation and neuronal cell death”, EP 855389, 29 Jul. 1998; Sakuma, Y., Tanaka, K., Suda, M., Yasoda, A., Natsui, K., Tanaka, I., Ushikubi, F., Narumiya, S., Segi, E., Sugimoto, Y., Ichikawa, A., Nakao, K. “Crucial involvement of the EP₄ subtype of prostaglandin E receptor in osteoclast formation by proinflammatory cytokines and lipopolysaccharide”, J. Bone Miner. Res., 2000, 15(2), 218-227).

PGE derivatives may also be used to treat vascular disease. PGE₁ is used clinically to lower blood pressure and improve vascular circulation. The role that the EP receptors have in the vasculature is now being determined. In a recent report of murine prostanoid receptor knockout (KO) mice, there was demonstrated a sexual dimorphism in EP-mediated blood pressure regulation (See: Audoly, L. P.; Tilley, J.; Key, M.; Nguyen, M.; Stock, J. L.; McNeish, J. D.; Koller, B. H.; Coffman, T. M. “Identification of specific EP receptors responsible for the hemodynamic effects of PGE₂”, Am. J Physiol., 1999, 46(3), H924-930). In female knockout mice, the EP₂ and EP₄ receptors mediated a vasodepressor response, whilst in the males it is the EP₁ receptor that mediates the vasodepressor response, which is opposed by the EP₃ receptor.

Additionally, antagonists of prostaglandin E₂ receptors, particularly EP₄ receptors have a diuretic effect and may be used for treating hypertension and premenstrual tension.

EP₄ and EP₂ selective ligands have the ability to lower intraocular pressure and thus are useful for the treatment of glaucoma. (See: U.S. Pat. Nos. 7,022,726; 7,015,243; and 6,977,260, incorporated herein by reference.) In addition, EP₄ selective ligands are useful for the prevention of neuronal cell death and EP₂ selective ligands can be used for protection against neuronal damage in the eye. In one aspect, the invention provides novel prostaglandin derivatives that can be used to lower ocular pressure and to act as protective agents for nerve cells, both for glaucoma as well as other diseases that are characterized by neuronal cell death.

### PGF Analogs

Analogs of PGF_{2α} are also useful for the treatment of a variety of medical conditions. PGF analogs can be used to treat bone disorders, such as osteoporosis. For example, one approach is to selectively activate the excitatory FP receptor as a means of reversing osteoporosis (see Hartke, J. R., Jankowsky, M. L., deLong, M. A., Soehner, M. E., Jee, W. S. S., Lundy, M. W. “Prostanoid FP agonists build bone in the ovariectomized rat”, J. Bone Min. Res., 1999, 14, S207; and Lundy, M. W., deLong, M. A., Combs, K. S., Gross, G. J., Soehner, M. E., Hartke, J. R. “Restoration of cancellous architecture and increased bone strength in aged osteopenic rats treated with fluprostenol”, J. Bone Min. Res., 1999, 14, S401.29).

FP ligands have also been proposed for the management of vascular diseases e.g., as vasorelaxants. PGF₁ analogs have also been disclosed for use in the treatment of diabetic and other forms of peripheral vascular disease (see Ueno, R., Oda, T. “Prostaglandins of the F series”, U.S. Pat. No. 5,770,759 issued Jun. 23, 1998).

FP receptor ligands may be used to treat ocular disorders such as glaucoma. (See: Liljebris, C. et al. “Derivatives of 17-Phenyl-18,19,20-trinorprostaglandin F2alpha Isopropyl Ester: Potential Antiglaucoma Agents”, Journal of Medicinal Chemistry, 1995, 38, 289-304; Babiole, M. et al. J. Ocular Pharmacology and Therapeutics, 17 (2), 2001 and references cited therein.)

PGF analogs can be used as sleep inducing agents. (See: Matsumura, H. “Prostaglandins and Sleep”, Saishin No to Shinkei Kagaku Shirizu, 1998, 10, 79-89.)

Other uses for the PGF derivatives include treating skin disorders; circulatory disorders, such as hypertension; gastrointestinal disorders; hair loss; respiratory disorders; and fertility control. More information regarding the biological effects of Prostaglandin F analogs is disclosed in the following references: “Molecular mechanisms of diverse actions of prostanoid receptors”, Biochimica et Biophysica Acta, 1259 (1995) 109-120; U.S. Pat. No. 3,776,938 issued Dec. 4, 1973 and U.S. Pat. No. 3,882,241 issued May 6, 1975; G.B. Patent No. 1,456,512 (1976) issued to Pfizer Inc.; Bundy, G. L.; Lincoln, F. H., “Synthesis of 17-Phenyl-18,19,20-trinor prostaglandins I. The PG1 Series”, Prostaglandins, 1975, 9, 1-4.; CRC Handbook of Eicosanoids: Prostaglandins and Related Lipids Vol. 1, Chemical and Biochemical Aspects, Parts A & B, A. L. Willis, eds., CRC Press (1987); Collins, P. W.; Djuric, S. W. “Synthesis of Therapeutically Useful Prostaglandin and Prostacyclin Analogs”, Chemical Reviews, 1993, 93, 1533-1564.

### PGD Analogs

PGD analogs can be used as sleep inducing agents. Sleep induction is generally thought to arise as a result of stimulation of the DP receptor (see Matsumura, H. “Prostaglandins and sleep”, Saishin No to Shinkei Kagaku Shirizu, 1998, 10, 79-89). Antagonists of the DP receptor may be used as anti-allergy agents. Prodrug forms of antiallergy agents are useful for ocular allergies.

### Other Prostaglandins

Other prostaglandins, such as PGA, in conjunction with phosphodiesterase inhibitors, may be used to enhance skin pigmentation. The enhancement of activity in the presence of diesterase inhibitors suggests that the EP₂ and the EP₄ receptors may be responsible. Alzheimer's Disease and renal salt wasting may be controlled by inhibition of the synthesis or activity of delta-12-PGJ₂. This prostaglandin is a known ligand of PPARγ.

However, none of the PGE, PGD, PGF, and PGA analogs disclosed above have their C₁ groups protected as a homotopically-symmetrical alcohol. In one embodiment, the invention provides prostaglandin derivatives wherein C₁ has been protected as an ester of a homotopically-symmetrical alcohol. Furthermore, prostaglandins either as free acids or as protected by other types of esters suffer from numerous drawbacks including poor water solubility and poor bioavailability. In addition, they are rapidly metabolized and removed from the desired tissue. In another embodiment, the invention provides prostaglandin derivatives that are more water soluble and/or less rapidly metabolized and exported than known prostaglandins and derivatives thereof.

### Prodrug Derivatives of Prostaglandins Using Alcohols with Homotopic Hydroxy Groups

The prostaglandin derivatives comprise a core prostaglandin bonded via the carboxylic acid at C₁ to a homotopically-symmetrical alcohol, R_{b}.

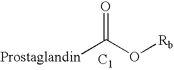

The prostaglandin is selected from the group of PGF analogs, PGE analogs, PGD analogs, PGA analogs, and PGB analogs.

The homotopic alcohol is selected from the group of tetraols, triols, and polyhydroxy alcohols (polyols), and cyclic polyols.

In one embodiment of the invention, the prostaglandin is a PGF analog or a PGE analog and the alcohol is a homotopic tetraol or triol.

In another embodiment of the invention, the prostaglandin is a PGF analog or a PGE analog and the alcohol is a homotopic monocyclic polyol.

The PGF analogs of this invention suitably have the general formula: 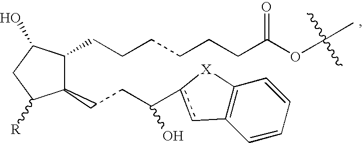
wherein the solid and dashed lines together indicate single or double bonds and the dashed line alone indicates single, double or triple bonds; the X indicates a CH₂, S, O, or SO₂ group; the wavy line indicates either an alpha or a beta configuration, or both in an admixture; and, R indicates an —H, an —OH, an —NHOH, an —NHOMe, or a halogen atom.

The PGE analogs of this invention suitable have the general formulae: 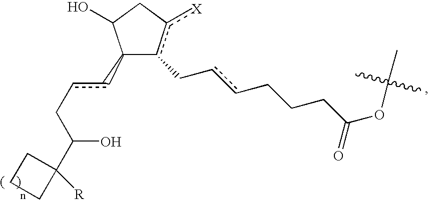
wherein X is selected from halogen, H, carbonyl (═O), and OH; R is a lower monovalent hydrocarbon group or lower heterogeneous group or an aromatic group or a heteroaromatic group, each of which may be substituted or unsubstituted; n is an integer from about 0 to about 4; and, the solid and dashed lines together indicate single or double bonds, or 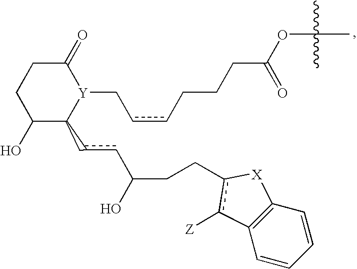
wherein X is selected from NH, S, CH₂, C═O, O, and SO₂; Y is N or CH; Z is a halogen, a lower monovalent hydrocarbon group or a lower heterogeneous group; and, the solid and dashed lines together indicate single or double bonds.

The PGD analogs of this invention suitably have the general formula: 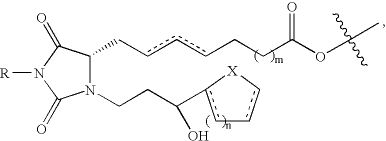
wherein X is selected from NR, S, O and SO₂; R is a lower monovalent hydrocarbon group or a lower heterogeneous group or an aromatic group or a heteroaromatic group, each of which may be substituted or unsubstituted; m and n are independently integers from about 0 to about 4; and, the solid and dashed lines together indicate single or double bonds.

The PGB analogs of this invention suitably have the general formula: 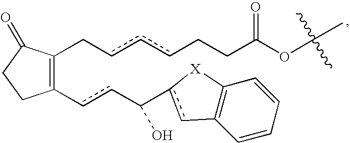
wherein X is selected from the group consisting of CH₂, NR, S, O and SO₂; R is selected from H, a methyl group, a hydrocarbon group, a substituted hydrocarbon group, a heterogeneous group, and a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group; and the solid and dashed lines together indicate single or double bonds.

The PGA analogs of this invention suitably have the general formula: 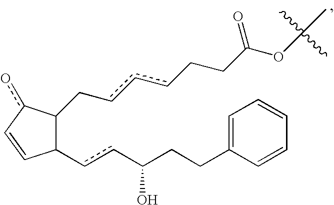
wherein the solid and dashed lines together indicate single or double bonds.

### Prodrug Derivatives of Ethacrynic Acid Using Alcohols with Homotonic Hydroxy Groups

The ethacrynic acid derivatives comprise a core phenoxyacetic acid or a cinnamic acid bonded via the carboxylic acid at C₁ to a homotopically-symmetrical alcohol, R_{b}.

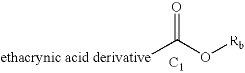

The ethacrynic is selected from the group of phenoxyacetic acids and cinnamic acids.

The phenoxyacetic acids of this invention suitably have the general formula: 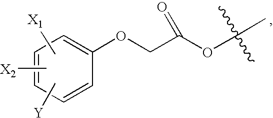
wherein X₁ and X₂ are independently selected from the group consisting of H, halogen, NR₁R₂, SR₁ and OR₁; R₁ and R₂ are each independently H, a lower monovalent hydrocarbon group or a lower heterogeneous group; and Y is a substituted or unsubstituted lower monovalent hydrocarbon group, lower heterogeneous group, aromatic group or heteroaromatic group.

The Cinnamic acids of this invention suitably have the general formula: 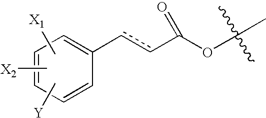
wherein X₁ and X₂ are independently selected from the group consisting of H, halogen, NR₁R₂, SR₁ and OR₁; R₁ and R₂ are each independently H, a lower monovalent hydrocarbon group or a lower heterogeneous group; Y is a substituted or unsubstituted lower monovalent hydrocarbon group, lower heterogeneous group, aromatic group or heteroaromatic group; and the solid and dashed lines together indicate a single or double bond.

### Prodrug Derivatives of Other Moieties Containing Acid Groups Using Alcohols with Homotopic Hydroxy Groups

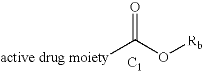

It is to be understood in the above cases that the C₁, or carboxylic acid moiety, is actually part of the drug, and not separate nor part of the homotopically-symmetrical alcohol, R_{b}. One of ordinary skill in the art will readily recognize drug and medicaments that contain the carboxylic acid moiety.

### Homotopically-Symmetrical Alcohols

The homotopically-symmetrical alcohols of the present invention comprise several groups, with the common characteristic of having all of their alcohols homotopic, that is, the groups are chemically-equivalent. They may most easily be divided into acyclic, alicyclic and polycyclic versions for discussion. One skilled in the art will recognize that other homotopically-symmetrically alcohols exist, and are also specifically contemplated in this invention.

The acyclic alcohol analogs of this invention suitably have the general formulae: 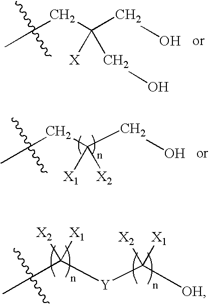
wherein each X, X₁ and X₂ are independently selected from the group consisting of H, a halogen atom, a hydroxymethyl group, a lower monovalent hydrocarbon group or a lower heterogeneous group, an alkoxymethyl group, an aryloxymethyl group, an amino group, a heterogeneous group, a carbocyclic group, a heterocyclic group, an aromatic group, a heteroaromatic group, a substituted carbocyclic group, a substituted heterocyclic group, a substituted aromatic group, or a substituted heteroaromatic group; Y is a bond, which may be single, double or triple, or is selected from the group consisting of (CX₁X₂)ₘ, O, NX₁, S, SO₂, or alternating or repeating units thereof; m and n are independently integers between zero and nine, particularly, zero and seven; and, the prostaglandin or other moiety is as described above with the caveat that the symmetry must be maintained for all free hydroxyls.

The alicyclic analogs of this invention suitably have the general formula: 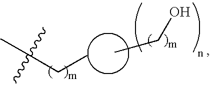
wherein the circle represents an alicyclic ring of from about 3 to about 7 member atoms, wherein the attachment point of the C₁ group is via a chain of member atoms consisting of m atoms, wherein m is an integer of from about zero to about 4 member atoms, and wherein there are n symmetrically-placed homotopic groups, wherein n is an integer from about one to about 7 groups. The ring and the member atoms of m may be substituted with other groups, with the caveat that the high-level symmetry must be maintained for all free hydroxyls. If substituted, the substitutions may be selected from a hydroxymethyl group, a hydroxyethyl group, an alkoxymethyl group, an aryloxymethyl group, a lower monovalent hydrocarbon group, a lower heterogeneous group, an amino group, a carbocyclic group, a heterocyclic group, an aromatic group, a heteroaromatic group, a substituted carbocyclic group, a substituted heterocyclic group, a substituted aromatic group, or a substituted heteroaromatic group.

Derivatives suitable for use in this invention may also be any optical isomer, and enantiomer of the above structures. Derivatives may be pharmaceutically-acceptable salts of the above structures, or any biohydrolyzable amides, esters, and imides of the above structures at positions other than the attachment of the symmetrical alcohol, or combinations thereof.

Compositions

This invention further relates to compositions comprising an ester derivative as described above as an active ingredient (hereinafter, component A). In particular, prostaglandin ester derivatives, ethacrynic acid ester derivatives, and cephalosporin ester derivatives are contemplated. The compositions can be pharmaceutical or cosmetic compositions, administered for treatment or prophylaxis of various conditions. Standard pharmaceutical formulation techniques are used, such as those disclosed in Remington's Pharmaceutical Sciences, Mack Publishing Company, Easton, Pa. (1990).

The composition further comprises component B) a carrier. “Carrier” means one or more compatible substances that are suitable for administration to a mammal. Carrier includes solid or liquid diluents, hydrotropes, surface-active agents, and encapsulating substances. “Compatible” means that the components of the composition are capable of being commingled with component A), and with each other, in a manner such that there is no interaction which would substantially reduce the efficacy of the composition under ordinary use situations. Carriers generally have sufficiently high purity and sufficiently low toxicity to render them suitable for administration to the mammal being treated. The carrier can be inert, or it can possess pharmaceutical benefits, cosmetic benefits, or both.

The choice of carrier for component B) depends on the route by which component A) will be administered and the form of the composition. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.

Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin; mannitol; and sorbitol. The amount of ingredient a) in the systemic composition is typically about 50 to about 90%.

Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of ingredient b) in the systemic composition is typically about 5 to about 10%.

Ingredient c) is a binder. Suitable binders for solid dosage forms include polyvinylpyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%.

Ingredient d) is a disintegrant. Suitable disintegrants for solid dosage forms include agar, alginic acid and the sodium salt thereof, effervescent mixtures, croscarmelose, crospovidone, sodium carboxymethyl starch, sodium starch glycolate, clays, and ion exchange resins. The amount of ingredient d) in the systemic composition is typically about 0.1 to about 10%.

Ingredient e) for solid dosage forms is a colorant such as an FD&C dye. The amount of ingredient e) in the systemic composition is typically about 0.005 to about 0.1%.

Ingredient f) for solid dosage forms is a flavor such as menthol, peppermint, and fruit flavors. The amount of ingredient f) in the systemic composition is typically about 0.1 to about 1.0%.

Ingredient g) for solid dosage forms is a sweetener such as aspartame and saccharin. The amount of ingredient g) in the systemic composition is typically about 0.001 to about 1%.

Ingredient h) is an antioxidant such as butylated hydroxyanisole (“BHA”), butylated hydroxytoluene (“BHT”), and vitamin E. The amount of ingredient h) in the systemic composition is typically about 0.1 to about 5%.

Ingredient j) is a preservative such as benzalkonium chloride, methyl paraben and sodium benzoate. The amount of ingredient j) in the systemic composition is typically about 0.01 to about 5%.

Ingredient k) for solid dosage forms is a glidant such as silicon dioxide. The amount of ingredient k) in the systemic composition is typically about 1 to about 5%.

Ingredient m) is a solvent, such as water, isotonic saline, ethyl oleate, alcohols such as ethanol, and phosphate buffer solutions. The amount of ingredient m) in the systemic composition is typically from about 0 to about 100%.

Ingredient n) is a suspending agent. Suitable suspending agents include AVICEL® RC-591 (from FMC Corporation of Philadelphia, Pa.) and sodium alginate. The amount of ingredient n) in the systemic composition is typically about 1 to about 8%.

Ingredient o) is a surfactant such as lecithin, polysorbate 80, and sodium lauryl sulfate, and the TWEENS® from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of ingredient o) in the systemic composition is typically about 0.1% to about 2%.

Although the amounts of components A) and B) in the systemic compositions will vary depending on the type of systemic composition prepared, the specific derivative selected for component A) and the ingredients of component B), in general, system compositions comprise 0.01% to 50% of component A) and 50 to 99.99% of component B).

Compositions for parenteral administration typically comprise A) 0.1 to 10% of the prodrug of the prostaglandin or other moiety and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50%, of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A), and component B) a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including time release and sustained release formulations) typically comprise component A), and B) a carrier comprising one or more a) diluents disclosed above in a capsule comprising gelatin. Granules typically comprise component A), and preferably further comprise k) glidants such as silicon dioxide to improve flow characteristics.

The selection of ingredients in the carrier for oral compositions depends on secondary considerations like taste, cost, and shelf stability, which are not critical for the purposes of this invention. One skilled in the art would know how to select appropriate ingredients without undue experimentation.

The solid compositions may also be coated by conventional methods, typically with pH or time-dependent coatings, such that A) the active prostaglandin or other moiety is released from its prodrug form in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise A) the prodrug of prostaglandin or other moiety and B) a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

In one embodiment of the invention, the prodrugs of the prostaglandin or other moiety are topically administered. Topical compositions that can be applied locally to the eye may be in any form known in the art, non-limiting examples of which include gelable drops, spray, ointment, or a sustained or non-sustained release unit placed in the conjunctival cul-du-sac of the eye.

Topical compositions that can be applied locally to the skin may be in any form including solutions, oils, creams, ointments, gels, lotions, shampoos, leave-on and rinse-out hair conditioners, milks, cleansers, moisturizers, sprays, skin patches, and the like. Topical compositions comprise: component A) the prodrug of the prostaglandin or other moiety described above and component B) a carrier. The carrier of the topical composition preferably aids penetration of the prodrug of the prostaglandin or other moiety into the skin. Component B) may further comprise one or more optional components.

The exact amounts of each component in the topical composition depend on various factors. The amount of component A) added to the topical composition is dependent on the IC₅₀ of component A), typically expressed in nanomolar (nM) units. For example, if the IC₅₀ of the free prostaglandin or other moiety is 1 nM, the amount of component A) will be from about 0.0001 to about 0.01%. If the IC₅₀ of the free prostaglandin or other moiety is 10 nM, the amount of component A) will be from about 0.001 to about 0.1%. If the IC₅₀ of the free prostaglandin or other moiety is 100 nM, the amount of component A) will be from about 0.01 to about 1.0%. If the IC₅₀ of the free prostaglandin or other moiety is 1000 nM, the amount of component A) will be 1.0 to 10%, particularly 1.0 to 5%. If the amount of component A) is outside the ranges specified above (i.e., either higher or lower), efficacy of the treatment may be reduced. IC₅₀ can be calculated according to the method in Reference Example 1, below. One skilled in the art would know how to calculate an IC₅₀. The remainder of the composition, up to 100%, is component B).

The amount of B) the carrier employed in conjunction with component A) is sufficient to provide a practical quantity of composition for administration per unit dose of the prodrug of the prostaglandin or other moiety. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B) the carrier may comprise a single ingredient or a combination of two or more ingredients. In the topical compositions, component B) comprises a topical carrier. Suitable topical carriers comprise one or more ingredients selected from the group consisting of phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and more particularly, phosphate buffered saline, isotonic water, deionized water, monofunctional alcohols and symmetrical alcohols.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Ingredient q) is an emollient. The amount of ingredient q) in a skin-based topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane.

Ingredient r) is a propellant. The amount of ingredient r) in the topical composition is typically about 0 to about 95%. Suitable propellants include propane, butane, isobutane, dimethyl ether, carbon dioxide, nitrous oxide, and combinations thereof.

Ingredient s) is a solvent. The amount of ingredient s) in the topical composition is typically about 0 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols.

Ingredient t) is a humectant. The amount of ingredient t) in the topical composition is typically 0 to 95%. Suitable humectants include glycerin, sorbitol, sodium 2-pyrrolidone-5-carboxylate, soluble collagen, dibutyl phthalate, gelatin, and combinations thereof. Specific humectants include glycerin.

Ingredient u) is a thickener. The amount of ingredient u) in the topical composition is typically about 0 to about 95%.

Ingredient v) is a powder. The amount of ingredient v) in the topical composition is typically 0 to 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. For ocular applications, specific powders include beta-cyclodextrin, hydroxypropyl cyclodextrin, and sodium polyacrylate. For gel dosing ocular formulations, sodium polyacrylate may be used.

Ingredient w) is a fragrance. The amount of ingredient w) in the topical composition is typically about 0 to about 0.5%, particularly, about 0.001 to about 0.1%. For ocular applications a fragrance is not generally used.

Ingredient x) is a pigment. Suitable pigments for skin applications include inorganic pigments, organic lake pigments, pearlescent pigments, and mixtures thereof. Inorganic pigments useful in this invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this invention include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. The amount of pigment in the topical composition is typically about 0 to about 10%. For ocular applications a pigment is not generally used.

In a particularly preferred embodiment of the invention, topical pharmaceutical compositions for ocular administration are prepared typically comprising component A) and B) a carrier, such as purified water, and one or more ingredients selected from the group consisting of y) sugars or sugar alcohols such as dextrans, particularly dextran 70, z) cellulose or a derivative thereof, aa) a salt, bb) disodium EDTA (Edetate disodium), and cc) a pH adjusting additive.

Examples of z) cellulose derivatives suitable for use in the topical pharmaceutical composition for ocular administration include sodium carboxymethylcellulose, ethylcellulose, methylcellulose, and hydroxypropyl-methylcellulose, particularly, hydroxypropyl-methylcellulose.

Examples of aa) salts suitable for use in the topical pharmaceutical composition for ocular administration include mono-, di- and trisodium phosphate, sodium chloride, potassium chloride, and combinations thereof.

Examples of cc) pH adjusting additives include HCl or NaOH in amounts sufficient to adjust the pH of the topical pharmaceutical composition for ocular administration to 6.8-7.5.

Component A) may be included in kits comprising component A), a systemic or topical composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the prodrug of the prostaglandin derivative, a composition, or both; and information, instructions, or both, regarding methods of application of the prodrug of the prostaglandin or other moiety, or of composition, preferably with the benefit of treating or preventing cosmetic and medical conditions in mammals (e.g., humans).

### Use of the Prodrug Derivatives and Compositions

This invention further relates to methods for treating medical and cosmetic conditions in mammals. The prodrug derivatives of this invention, and compositions containing these derivatives, can be used to treat subjects suffering from many medical and cosmetic conditions. Generally, the method comprises administering to a mammal in need of treatment, a prodrug of a prostaglandin derivative or other moiety (or composition) described above.

The conditions that can be treated depend on the type of derivative, i.e., the receptor or receptors for which the derivative has affinity and whether the derivative is an agonist or antagonist for the receptor, or the ability of the compound to impact the organism in a desirable way, when the mechanism of action of the moiety is unclear.

Naturally occurring PGF₂, has the formula:

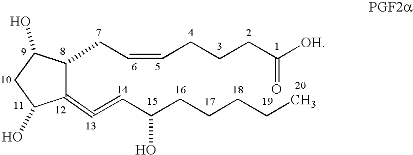

A typical prodrug PGF derivative according to this invention has the formula:

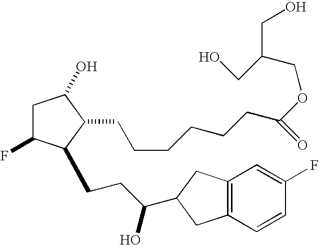

Naturally occurring PGF_{2α} has a bond in the alpha configuration at position C₁₁. The PGF derivative has a bond in the beta configuration at position C₁₁. The difference in substitution and bond configuration at position C₁₁ indicates that the novel PGF derivative is a potential antagonist.

Selectivity of prostaglandin derivatives toward different receptors can be controlled by varying certain groups at specified positions in the prostaglandin derivative. (Numbering is based on that of naturally-occurring prostaglandins, so they must be translated to the appropriate position on the derivative.) A 9-ketone or other sp²-hybridized group is suitable for the derivative to have selectivity toward the EP receptors. A 9-hydroxyl and a 15-hydroxyl are suitable for the derivative to have selectivity toward the FP receptor. Different groups may be present at position 11, but a hydroxyl group is particularly suitable for PGF derivatives. An 11-ketone or other sp²-hybridized group is suitable for the derivative to have selectivity toward the DP receptor. A hydrophobic cyclopentyl ring is suitable to confer selectivity to the TP receptor, and a 5,6-alkene is also suitable.

One skilled in the art would, without undue experimentation, be able to determine the receptor for which a derivative has affinity and whether the derivative is an agonist or antagonist, using the above guidelines for determining affinity. One commercially-available test for agonism and antagonism at the FP receptor is the antifertility assay offered by PanLabs of Seattle, Wash.

### PGE Derivatives

EP₁ agonists can be used to treat bone disorders such as osteoporosis, vascular diseases such as high blood pressure and poor vascular circulation, sexual dysfunction such as erectile dysfunction and women's sexual arousal dysfunction. PGE, agonists can also be used to enhance skin pigmentation. EP₁ antagonists can be used to treat pain.

EP₂ agonists can be used to treat glaucoma, asthma and bone disorders such as osteoporosis. EP₂ agonists can be used to inhibit cell migration and protect against neuronal damage in the eye. EP₂ agonists can also be used to enhance skin pigmentation. EP₂ antagonists have a diuretic effect and may be used to treat hypertension and premenstrual tension.

EP₃ agonists can be used to treat arthritis, bone disorders such as osteoporosis, vascular disease such as high blood pressure and poor vascular circulation. EP₃ agonists can also be used to enhance uterine contractions and inhibit gastric acid secretion. EP₃ agonists can also be used to prevent or treat, or both, hepatic diseases, renal diseases, pancreatitis, myocardial infarct, and gastric disturbances such as ulcers. EP₃ antagonists can be used to control blood pressure.

EP₄ agonists can be used to treat glaucoma, arthritis, and bone disorders such as osteoporosis, vascular disease such as high blood pressure and poor vascular circulation, and asthma. EP₄ agonists can also be used to inhibit cell migration and prevent neuronal cell death, especially in ocular formulations. EP₄ agonists can also be used to enhance skin pigmentation. EP₄ antagonists have a diuretic effect and can be used to treat hypertension and premenstrual tension.

PGE analogs used to treat the above conditions include those selected from the group consisting of: 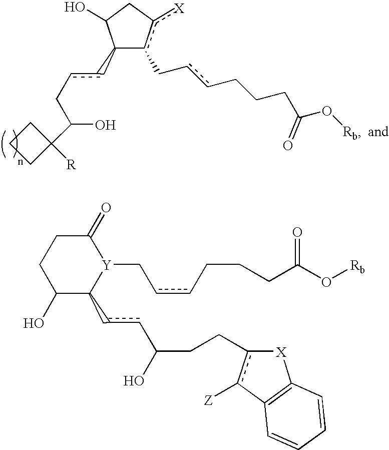
wherein the variables n, X, R, Y, Z and R_{b} are as defined above, and wherein the solid and dashed lines together indicate single or double bonds.

Other suitable derivatives include optical isomers of the structures described above, as well as, diastereomers of the above structures, enantiomers of the above structures, pharmaceutically-acceptable salts of the above structures, biohydrolyzable amides of the above structures, biohydrolyzable esters of the above structures, and biohydrolyzable imides of the above structures.

### PGF Derivatives

FP agonists can be used to treat ocular disorders such as increased intraocular pressure, glaucoma, skin disorders, bone disorders such as osteoporosis, circulatory disorders such as hypertension, gastrointestinal disorders, hair loss, and respiratory disorders. FP agonists can also be used for fertility control, to manage vascular diseases such as diabetic and other forms of peripheral vascular disease, to induce labor, and as nasal decongestants. FP antagonists can be used to prevent premature labor and to prevent hyper-pigmentation of the skin.

PGF derivatives suitable to treat the above conditions include those having the following structure: 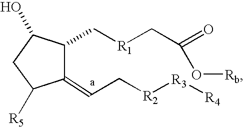
wherein R¹ is a divalent group selected from the group consisting of a hydrocarbon group, a substituted hydrocarbon group, a heterogeneous group, and a substituted heterogeneous group, with the proviso that when R¹ is a heterogeneous group, R¹ has only one heteroatom selected from the group consisting of oxygen, sulfur, and nitrogen; R² is a divalent group selected from the group consisting of —C(O)—, —C(R⁶)(OR⁷)— and —CF₂—; R³ is a divalent group having the formula —(CD(D))ₚ-X—(CD(D))_{q}-, wherein p is an integer from 0 to 3 and q is an integer from 0 to 3, X is selected from the group consisting of an oxygen atom, a divalent hydrocarbon group, a sulfur atom, SO, SO₂, and ND, and D is selected from the group consisting of a hydrogen atom, a monovalent hydrocarbon group of 1 to 4 carbon atoms, and a monovalent heterogeneous group of 1 to 4 member atoms; R⁴ is selected from the group consisting of a methyl group, a carbocyclic group, a substituted carbocyclic group, a heterocyclic group, a substituted heterocyclic group, an aromatic group, a substituted aromatic group, a heteroaromatic group, and a substituted heteroaromatic group; R⁵ is selected from the group consisting of a hydrogen atom, a halogen atom, NHR⁶, a carbonyl group and OR⁶; and R⁶ and R⁷ are independently selected from the group consisting of a hydrogen atom, a monovalent hydrocarbon group of 1 to 4 carbon atoms, and a monovalent heterogeneous group of 1 to 4 member atoms; and bond a is selected from the group consisting of a triple bond, a single bond and a double bond.

Other suitable derivatives include optical isomers of the structure described above, as well as, diastereomers of the above structure, enantiomers of the above structure, pharmaceutically-acceptable salts of the above structure, biohydrolyzable amides of the above structure, biohydrolyzable esters of the above structure, biohydrolyzable imides of the above structure and combinations thereof.

### PGD Derivatives

PGD agonists can be used to induce sleep, e.g. for treating sleeping disorders such as insomnia. PGD antagonists can be used to treat allergies, especially ocular allergies. PGD derivatives suitable to treat the above conditions include those having a structure selected from the group consisting of: 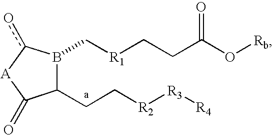
wherein dashed lines, bond a, R¹, R², R³, R⁴, R⁶, R⁷ and R_{b} are as described above and, wherein A is selected from the group of CH₂ and NR⁶, and B is selected from the group —CH or N.

Other suitable derivatives include optical isomers of the structure described above, as well as, diastereomers of the above structure, enantiomers of the above structure, pharmaceutically-acceptable salts of the above structure, biohydrolyzable amides of the above structure, biohydrolyzable esters of the above structure, biohydrolyzable imides of the above structure and combinations thereof.

### Dosage of Prostaglandin Derivatives

The dosage of the prostaglandin derivative administered depends on a variety of factors, including the method of administration. For systemic administration, (e.g., oral, rectal, sublingual, buccal, or parenteral), typically, 0.5 mg to 300 mg, particularly 0.5 mg to 100 mg, more particularly 0.1 mg to 10 mg, of a prostaglandin derivative described above is administered per day. These dosage ranges are merely exemplary, and daily administration can be adjusted depending on various factors. The specific dosage of the prostaglandin derivative to be administered, as well as the duration of treatment, the condition being treated, and whether the treatment is topical or systemic are interdependent. The dosage and treatment regimen will also depend upon such factors as the condition being treated, the specific prostaglandin derivative used, the treatment indication, the efficacy of the compound, the personal attributes of the subject (such as, for example, weight, age, sex, and medical condition of the subject), compliance with the treatment regimen, and the presence and severity of any side effects of the treatment.

Systemic administration (e.g., parenteral, oral, sublingual, buccal) is preferably carried out one to four times per day for the duration of treatment. Systemic administration is suitable for treating vascular disease such high blood pressure and poor vascular circulation.

For topical administration (e.g., local application on the skin, ocular, nasal, liposome delivery systems, or iontophoresis), the topical composition is typically administered once or twice per day for the duration of treatment. For some conditions, 6 to 12 weeks is sufficient. Topical administration is suitable in treating conditions such as nasal congestion, allergies, eyelash and hair loss, treating skin conditions (e.g., enhancing skin growth and skin pigmentation), and for treating all manner of ocular disorders such as glaucoma, ocular allergies, ocular infection, ocular neuronal protection, hyperemia or redness of the eyes, and for local vasodilatation

It should be recognized that different combinations of a homotopically-symmetrical alcohol and a biologically-active drug-moiety containing a carboxylic acid will have differing susceptibility to hydrolysis by various enzymes. Matching the hydrolysis rate to the enzymatic profile of the target tissue and the desired release rates will ensure optimal dosing and delivery. In addition, the solubility and lipophilicity of a particular homotopically-symmetrical alcohol ester will influence the partitioning of the compound into the tissue wherein hydrolysis occurs. Reasonable methods for estimating the lipophilicity of compounds are well known to those skilled in the art. However, in vitro methods for: 1) ranking the susceptibility to hydrolysis and, 2) determining the optimal dosing concentrations in a particular tissue of interest are not well established and require development of methods which are relevant to the tissue of interest.

In one aspect of this invention, butyrylcholinesterase is specifically used. Butyrylcholinesterase is the only corneal enzyme shown to cleave ester-containing compounds relevant to glaucoma treatment, combined with a method for ranking the rates of hydrolysis based on kinetic data. This method can be used to determine: 1) the susceptibility to hydrolysis by butyrylcholinesterase; and 2) the concentration of each symmetrical alcohol required to release an amount of carboxylic acid required to be maximally effective in an in vivo model of the specific disease state.

In an additional aspect of this invention, an advantage of this invention is the use of corneal epithelial cell homogenate, combined with a method for ranking the rates of hydrolysis based on kinetic data. This method can be used to determine: 1) the susceptibility to hydrolysis by enzymes present in the human corneal epithelium; and 2) the concentration of each homotopically-symmetrical alcohol required to release an amount of biologically-active moiety required to be maximally effective in an in vivo model of the specific disease state. To those skilled in the art, both the butyrylcholinesterase and corneal epithelial homogenate assays are easily adapted to multiwell plate formats, enabling very high throughput kinetic data generation.

The most appropriate candidates can then be advanced to testing in in vivo systems. In vivo pharmacological activity for glaucoma can be determined using assays designed to test the ability of the subject compounds to decrease intraocular pressure. Examples of such assays are described in the following reference, incorporated herein: Liljebris, C.; Selen, G.; Resul, B.; Stemschantz, J.; Hacksell, U. “Derivatives of 17-Phenyl-18,19,20-trinorprostaglandin Isopropyl Ester: Potential Antiglaucoma Agents”, Journal of Medicinal Chemistry, Vol. 38 No. 2 (1995), 289-304.

It is recognized that each drug and disease state will have its own unique requirements for dosing and delivery, and that one skilled in the art may modify the guidelines described herein. Combinations which are rapidly hydrolyzed may present the opportunity to lower the dosing concentration while delivering sufficient quantities of the biologically-active drug moiety to the target tissue prior to being washed out of the eye. Compounds which partition into the cornea and are more slowly hydrolyzed may allow an extended period of release and possible reduction of dosing interval. In one embodiment, the invention provides a method for determining the optimal homotopically-symmetrical alcohol esters required for the release of their biologically-active moieties under the conditions described. Another embodiment of the invention allows for the in vitro determination of the optimal concentration of said homotopically-symmetrical alcohol esters required for the release of biologically-active moiety, over a pre-defined time period, to result in reduced intraocular pressure in an in vivo model of glaucoma.

### EXAMPLES

These examples are intended to illustrate the invention to those skilled in the art and should not be interpreted as limiting the scope of the invention set forth in the claims.

The following abbreviations are used in the examples.

“DBU” means 1,8-diazabicyclo[5.4.0.]-undec-7-ene

“DCC” means dicyclocarbodiimide

“DMAP” means 4-dimethylaminopyridine

“DMF” means N,N-dimethylformamide

“EDC” means N-(3-dimethylaminopropyl)-N′-ethylcarbodiimide hydrochloride

“Et” means an ethyl group.

“EtOAc” means ethyl acetate.

“LAH” means lithium aluminum hydride.

“Me” means a methyl group.

“MeOH” means methanol.

“PCC” means pyridinium chlorochromate.

“TBAF” means tetra-N-butyl ammonium fluoride

“TBDMS” means tert-butyldimethylsilyl.

“TBDMSOTf” means tert-butyldimethylsilyl triflate.

“TBDMSCl” means tert-butyl dimethylsilyl chloride.

“TBS” means tert-butyldimethylsilyl”

“THF” means tetrahydrofuran.

“TLC” means thin layer chromatography.

“TMS” means trimethylsilyl.

### Example 1

Preparation of (Z)-3-hydroxy-2,2-bis(hydroxymethyl)propyl 7-((1R,2R,3S,5S)-5-(tert-butyldimethylsilyloxy)-2-((R)-3-(tert-butyldimethylsilyloxy)-3-(2,3-dihydro-1H-inden-2-yl)propyl)-3-fluorocyclopentyl)hept-5-enoate (E1b):

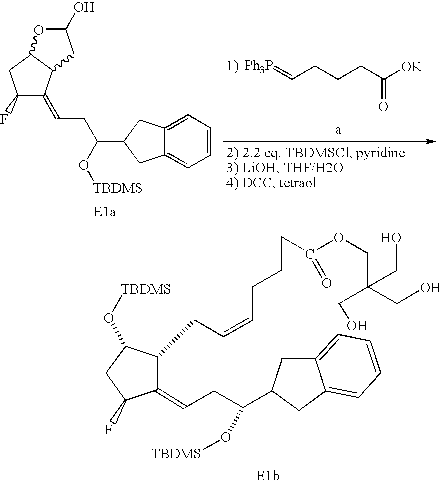

In a round bottom flask under argon the Wittig salt (a) (2.2 equiv.) is added to THF, and cooled to −78° C. Sodium hexamethyldisilazide (4.4 equiv.) is then added in one portion and the reaction is stirred for 15 minutes at −78° C. The solution is then warmed to 0° C. for two hours. The reaction mixture is cooled to −78° C. and the lactol E1a in THF is added over 10 minutes. E1a is prepared from Corey Aldehyde in a manner analogous to that taught in PCT Publication No. WO 98/20880. The solution is stirred at −78° C. for 1 hour and then allowed to warm to room temperature and stirred an additional 17 hours. The reaction mixture is quenched with water and the THF is removed under reduced pressure. The residue is dissolved in EtOAc/hexane and washed two times with 1N HCl. The organic layer is dried with Na₂SO₄) and solvent is removed under reduced pressure.

To a solution of the crude free acid (1 equiv.) in THF/pyridine at 0° C. is added TBDMSCl (2.3 equiv.) in one portion. The reaction mixture is stirred for 2.5 hours at room temperature and then quenched with water. The solution is poured into CH₂Cl₂ and the aqueous layer is acidified to a pH of 1. The aqueous layer is re-extracted with CH₂Cl₂ and organics are combined. The organic layer is dried (Na₂SO₄) and concentrated. The residue is chromatographed on SiO₂ (10% EtOAc/hexanes) to provide the intermediate as a colorless oil. To a solution of that compound and THF/water is added, at 0° C., over 10 min, LiOH (10 equiv.). The reaction is monitored by TLC (4% EtOAc/hexanes/0.1% formic acid) until complete. The solution is added to 5% EtOAc/hexanes and brine and the aqueous layer is acidified to pH˜1.0, then the mixture is chromatographed on SiO₂ (5% EtOAc/hexanes/0.1% formic acid) to provide the 9,15-bis silyl free acid as a colorless oil. This product is treated with DCC and an excess of the tetraol alcohol. The reaction is stirred at 25° C. overnight. The reaction is then quenched with water. The organic layer is washed with brine, dried over MgSO₄, and concentrated in vacuo to give a crude product which is purified by flash chromatography on silica gel (hexanes/THF gradient) to give (Z)-3-hydroxy-2,2-bis(hydroxymethyl)propyl 7-((1R,2R,3S,5S)-5-(tert-butyldimethyl-silyloxy)-2-((R)-3-(tert-butyldimethylsilyloxy)-3-(2,3-dihydro-1H-inden-2-yl)propyl)-3-fluorocyclopentyl)hept-5-enoate (E1b).

### Example 2

(Z)-3-hydroxy-2,2-bis(hydroxymethyl)propyl 7-((1R,2R,3S,5S)-2-((R)-3-(2,3-dihydro-1H-inden-2-yl)-3-hydroxypropyl)-3-fluoro-5-hydroxycyclopentyl)hept-5-enoate (E2a):

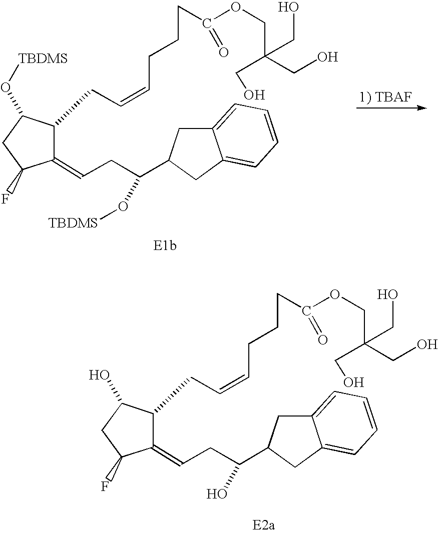

A mixture of (E1b) and TBAF (2.3 equiv.) in THF is stirred at 0° C. for 8 hours. The progress of the reaction is monitored by TLC. The reaction is then quenched with water and CH₂Cl₂. The organic layer is washed with brine, dried over MgSO₄, and concentrated in vacuo to give a crude product which is purified by flash chromatography on silica gel (hexanes/THF gradient) to give (Z)-3-hydroxy-2,2-bis(hydroxymethyl)propyl 7-((1R,2R,3S,5S)-2-((R)-3-(2,3-dihydro-1H-inden-2-yl)-3-hydroxypropyl)-3-fluoro-5-hydroxycyclopentyl)hept-5-enoate, (E2a)

### Examples 3-14

Compounds 3-14 are made by using the procedure set forth in Examples 1 and 2 and substituting the appropriate starting materials.

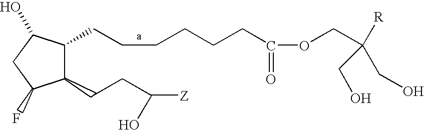

| Example | R | a | Z |
|---|---|---|---|
| 3 | Me | CH₂CH₂ | 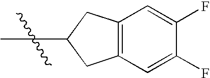 |
| 4 | Et | CH═CH (cis) | 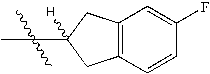 |
| 5 | CH₂OH | 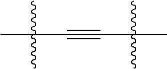 | 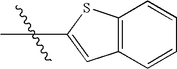 |
| 6 | H | —CH═C═CH— | 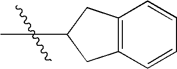 |
| 7 | Me | CH₂CH₂ | 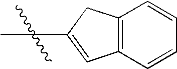 |
| 8 | CH₂OH | CH═CH (cis) | 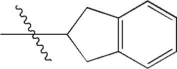 |
| 9 | Et | CH═CH (cis) | 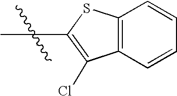 |
| 10 | Me | CH₂CH₂ | 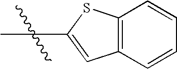 |
| 11 | iPr | CH═CH (trans) | 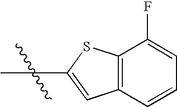 |
| 12 | H | CH═CH (cis) | 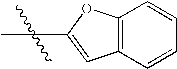 |
| 13 | Me | CH₂CH₂ | 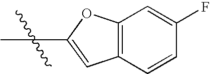 |
| 14 | CH₂OMe | CH═CH (trans) | 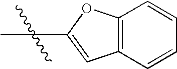 |

### Example 15

Preparation of 3-hydroxy-2,2-bis(hydroxymethyl)propyl 7-((1R,2R,3R,5S)-2-((R)-3-(benzo[b]thiophen-2-yl)-3-(tert-butyldimethyl silyloxy)propyl)-3,5-bis(tert-butyldimethyl silyloxy)cyclopentyl) heptanoate (E15b):

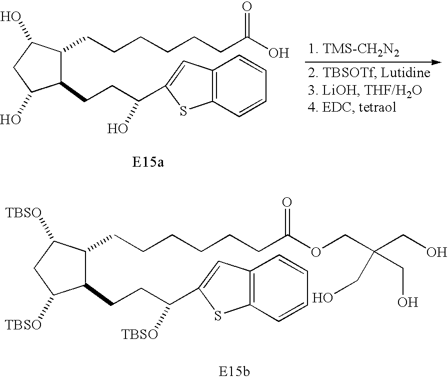

To acid E15a in MeOH cooled to 0° C. is added trimethylsilyldiazomethane (2 equiv.) and the solution is warmed and stirred at room temperature for 2 hours. The solvents are evaporated and the residue is chromatographed on SiO₂ (EtOAc/hexanes gradient) to provide the intermediate methyl ester.

Lutidine (3.8 equiv.) and tert-butyldimethylsilyl triflate (3.5 equiv.) are added to a cooled (0° C.) solution of the methyl ester in CH₂Cl₂ and the solution is warmed to room temperature and stirred for 12 hours. Then the mixture is poured into a solution of NH₄Cl₍ₛₐₜ₎/HCl (1 N) and EtOAc and extracted with EtOAc. The combined organics are dried (Na₂SO₄), filtered, evaporated and chromatographed on SiO₂ (5% EtOAc/hexanes) to give the TBS-protected, methyl ester derivative as a colorless oil.

To a solution of this intermediate in THF/MeOH/H₂O is added LiOH (5 equiv.) and the mixture is stirred for 12 hours (or until completion of the reaction is indicated by TLC) at room temperature. The solvents are evaporated and the mixture is added to a solution of NH₄Cl₍ₛₐₜ₎/HCl (1 N) and EtOAc and extracted with EtOAc. The organics are dried (Na₂SO₄), filtered, evaporated and chromatographed on SiO₂ (10% MeOH/CH₂Cl₂) to provide the corresponding TBS-protected acid.

This compound is treated with EDC and an excess of the tetraol alcohol and stirred overnight at 25° C. The reaction is washed with NH₄Cl₍ₛₐₜ₎, extracted with EtOAc, dried (Na₂SO₄), filtered, evaporated. Column chromatography (SiO₂, EtOAc/hexanes) gives 3-hydroxy-2,2-bis(hydroxymethyl)propyl7-((1R,2R,3R,5S)-2-((R)-3-(benzo[b]thiophen-2-yl)-3-(tert-butyldimethylsilyloxy)propyl)-3,5-bis(tert-butyldimethylsilyl oxy)cyclopentyl)heptanoate (E15b).

### Example 16

Preparation of 3-hydroxy-2,2-bis(hydroxymethyl)propyl 7-((1R,2R,3R,5S)-2-((R)-3-(benzo[b]thiophen-2-yl)-3-hydroxypropyl)-3,5-dihydroxycyclopentyl)heptanoate (E16a)

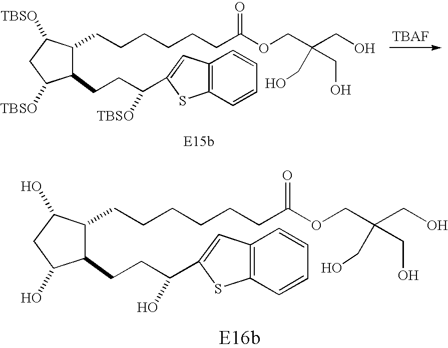

To a cooled solution (0° C.) of E15b in THF is added TBAF (8.0 equiv.). The mixture is warmed and stirred at room temperature for 2 hours or until TLC indicates completion of the reaction. The solvents are evaporated and the crude reaction mixture is chromatographed (hexanes/THF gradient) to give 3-hydroxy-2,2-bis(hydroxy methyl)propyl 7-((1R,2RS,3R,5S)-2-((R)-3-(benzo[b]thiophen-2-yl)-3-hydroxypropyl)-3,5 dihydroxy cyclo pentyl)heptanoate (E16a).

### Examples 17-35

Compounds 17-35 are made by using the procedure set forth in Examples 15 and 16 and substituting the appropriate starting materials.

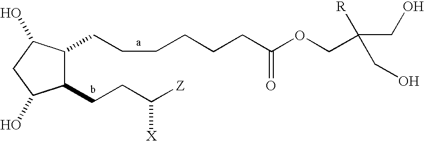

| Example | R | a | b | X | Z |
|---|---|---|---|---|---|
| 17, 18, 19 | H, Me, Et | CH₂CH₂ | CH₂CH₂ | OH | 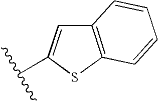 |
| 20, 21, 22, 23 | H, Me, Et, CH₂OH | CH═CH (cis) | CH₂CH₂ | OH | 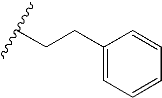 |
| 24, 25, 26, 27 | H, Me, Et, CH₂OH | CH═CH | CH₂CH₂ | O | —(CH₂)₆CH₃ |
| 28, 29, 30, 31 | H, Me, Et, CH₂OH | CH═CH (cis) | CH═CH (trans) | OH | 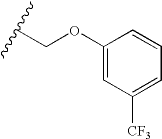 |
| 32, 33, 34, 35 | H, Me, Et, CH₂OH | CH═CH (trans) | CH═CH (trans) | OH | 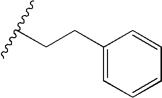 |

### Examples 36-45

Compounds 36-45 are made using the procedure set forth in Examples 15 and 16 and substituting the appropriate starting materials.

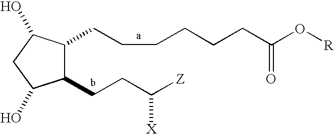

| Example | R | | a | b | X | Z |
|---|---|---|---|---|---|---|
| 36, 37 | 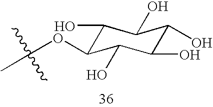 | 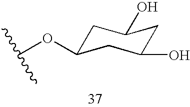 | CH₂CH₂ | CH₂CH₂ | OH | 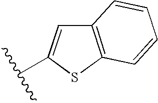 |
| 38, 39 | 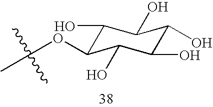 | 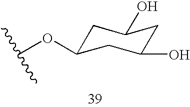 | CH═CH (cis) | CH₂CH₂ | OH | 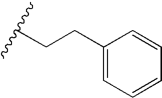 |
| 40, 41 | 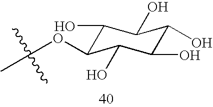 | 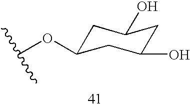 | CH═CH (trans) | CH₂CH₂ | ═O | —(CH₂)₆CH₃ |
| 42, 43 | 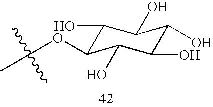 | 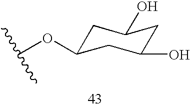 | CH═CH (cis) | CH═CH (trans) | OH | 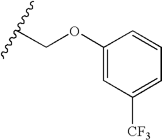 |
| 44, 45 | 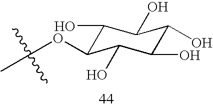 | 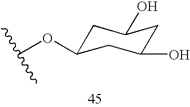 | CH═CH (cis) | CH═CH (trans) | OH | 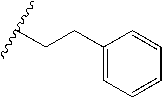 |

### Example 46

### Preparation of 3-hydroxypropyl 7-((1R,2R,3R,5S)-2-((R)-3-(benzo[b]thiophen-2-yl)-3-hydroxypropyl)-3,5-dihydroxycyclopentyl)heptanoate (E46).

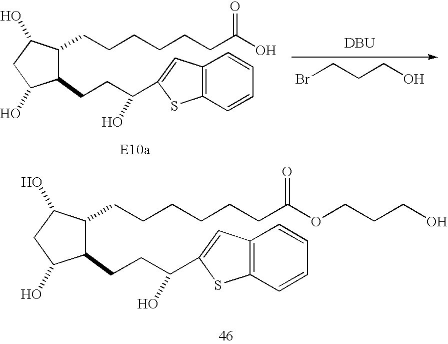

To acid E10a in acetone at 0° C. is added DBU (10 equiv.) and the mixture is stirred for one hour. Then 3-bromo-1-propanol (10 equiv.) is added and the mixture is warmed to room temperature and stirred for 12 hours. The mixture is poured into NH₄Cl₍ₛₐₜ₎ and extracted with EtOAc. The organics are dried (Na₂SO₄), filtered, and evaporated to give crude E46. Column chromatography (SiO₂, 10% MeOH, CH₂Cl₂) gives pure 3-hydroxypropyl 7-((1R,2R,3R,5S)-2-((R)-3-(benzo[b]thiophen-2-yl)-3-hydroxypropyl)-3,5-dihydroxycyclopentyl)heptanoate (E46).

### Examples 47-105

Compounds 47-105 are made using the procedure set forth in Examples 15, 16 and 46 and substituting the appropriate starting materials.

| | | | | |
|---|---|---|---|---|
| R | a | b | X | Z |

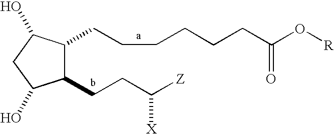

| | | | | |
|---|---|---|---|---|
| 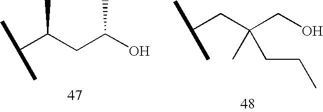 | CH₂CH₂ | CH₂CH₂ | OH | 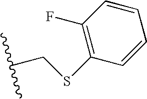 |
| 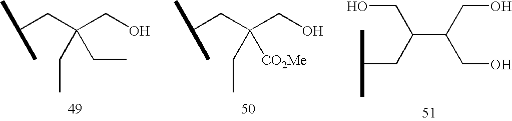 | | | | |
| 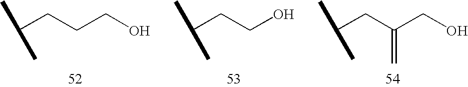 | CH═CH (cis) | CH₂CH₂ | OH | 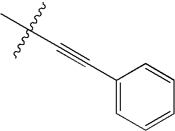 |
| 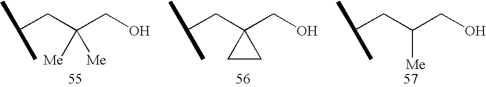 | | | | |
| 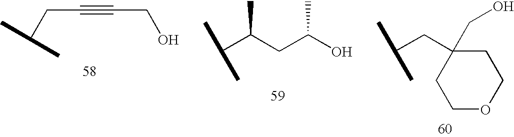 | | | | |
| 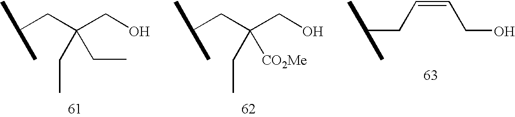 | | | | |
| 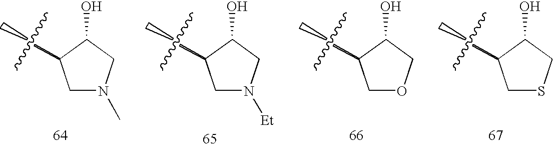 | —C≡C— | CH═CH (trans) | F | 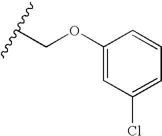 |
| 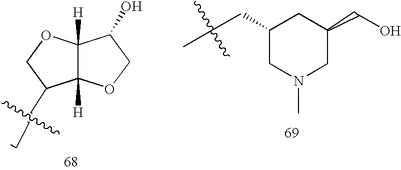 | | | | |
| 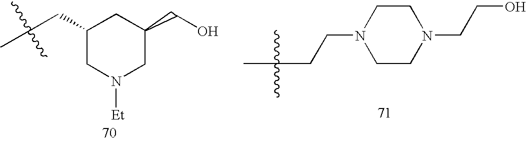 | | | | |
| 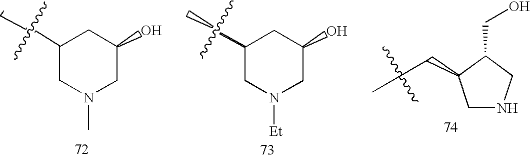 | | | | |
| 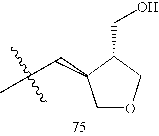 | | | | |
| 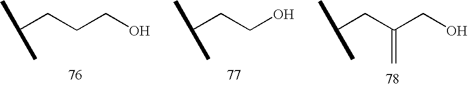 | CH═CH (cis) | CH═CH | OH | 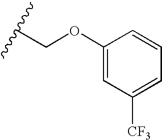 |
| 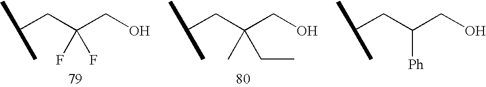 | | | | |
| 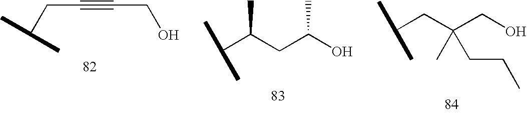 | | | | |
| 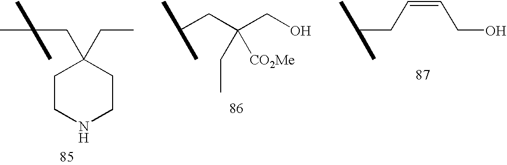 | | | | |
| 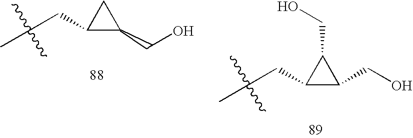 | CH═CH (trans) | CH═CH (trans) | OH | 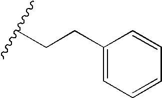 |
| 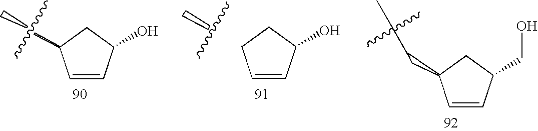 | | | | |
| 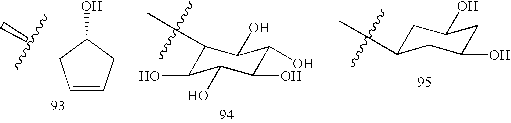 | | | | |
| 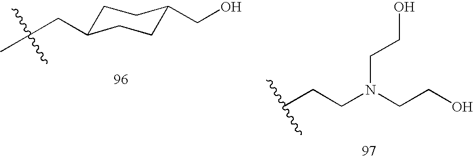 | | | | |
| 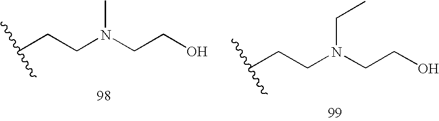 | | | | |

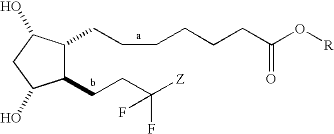

| | | | | |
|---|---|---|---|---|
| 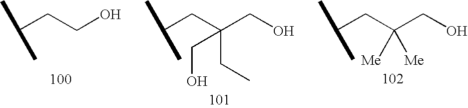 | CH═CH (cis) | CH₂CH₂ | n/a | 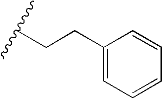 |
| 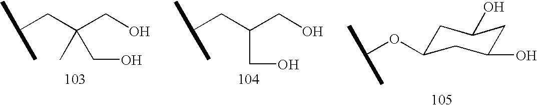 | | | | |

Examples 1-105 show that FP-selective agonists and antagonists can be prepared according to this invention.

### Examples 106-117

Compounds 106-117 are made using the procedure set forth in Examples 15, 16 and 46 and substituting the appropriate starting materials.

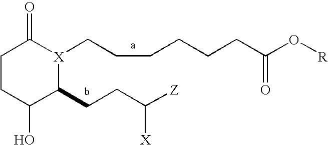

| Example | R | | a | b | X | Z |
|---|---|---|---|---|---|---|
| 106; 107; 108; 109 | 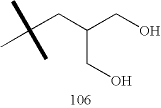 | 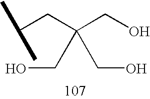 | CH₂CH₂ | CH₂CH₂ | OH | 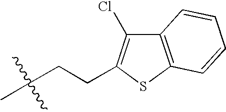 |
| | 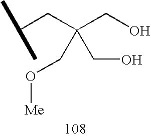 | 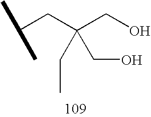 | | | | |
| 110, 111 | 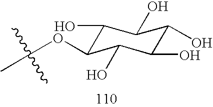 | 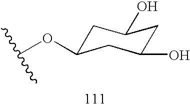 | CH═CH (cis) | CH₂CH₂ | OH | 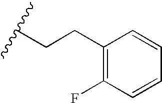 |
| 112, 113 | 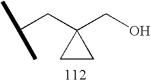 | 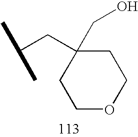 | —C≡C— | CH₂CH₂ | N | 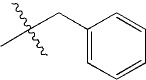 |
| 114, 115 | 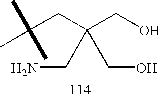 | 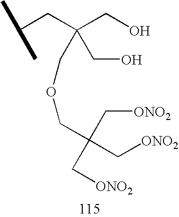 | CH═CH (trans) | CH═CH (trans) | OH | 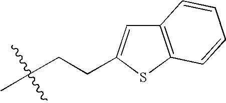 |
| 116, 117 | 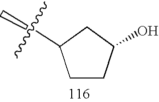 | 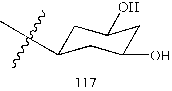 | CH═CH (cis) | CH═CH (trans) | N | 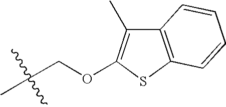 |

### Example 118

Preparation of (Z)-7-((1R,2R,3R)-3-(tert-butyldimethylsilyloxy)-2-((S,E)-3-(tert-butyldimethylsilyloxy)-5-(3-(methoxymethyl)phenyl)pent-1-enyl)-5-oxocyclo pentyl)hept-5-enoic acid (E118b):

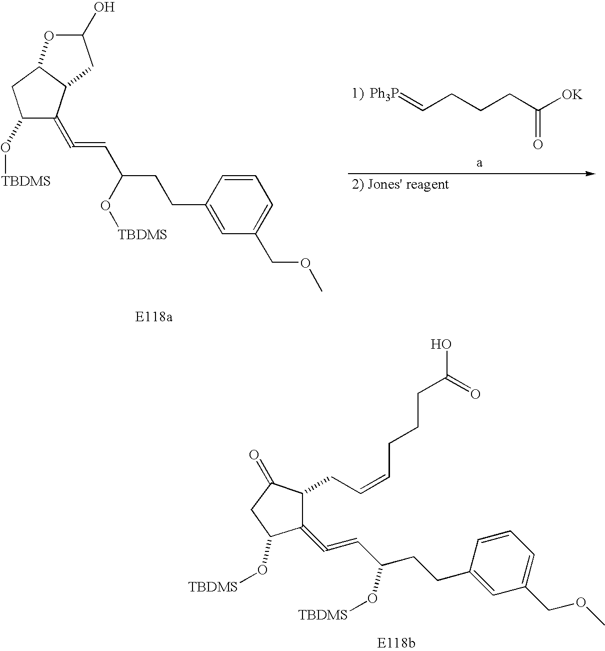

In a round bottom flask under argon the Wittig salt (a, Aldrich Chemical Company, Milwaukee, Wis.) (2.2 equiv.) is added to THF, and cooled to −78° C. Sodium hexamethyldisilazide (4.4 equiv.) is then added in one portion and the reaction is stirred for 15 minutes at −78° C. The solution is then warmed to 0° C. for two hours. The reaction mixture is then cooled again to −78° C. and the lactol E118a, in THF is added over 10 minutes. E118a is prepared from Corey Aldehyde in a manner analogous to that taught in U.S. Pat. No. 6,048,895 issued Apr. 11, 2000. The solution is stirred at −78° C. for 1 hour and then allowed to warm to room temperature and stirred an additional 17 hours. The reaction mixture is quenched with water and the THF is removed under reduced pressure. The residue is brought up in EtOAc/hexane and washed twice with 1N HCl. The organic layer is dried with (Na₂SO₄) and solvent is removed under reduced pressure. The residue is taken up in acetone and Jones' reagent is added dropwise. When the green color persists, the reaction is checked by TLC, and then is extracted with EtOAc/hexane and washed twice with brine. The organic layer is dried with (Na₂SO₄) and solvent is removed under reduced pressure. The residue is chromatographed on SiO₂ (10% EtOAc/hexanes) to provide the free acid as a yellow oil.

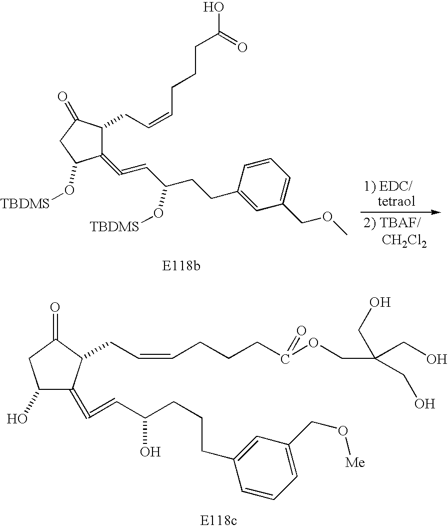

b. 17-(3-methoxy-methylphenyl)-17-trinor-PGF_{2α} (E118c): Using the methods of Examples 15 and 16, and starting with E118b and using 2,2-bis(hydroxymethyl)propane-1,3-diol as the tertrol, compound E118c is produced.

### Examples 119-124

Compounds 119-124 are made using the procedure set forth in Example 118 and substituting the appropriate starting materials.

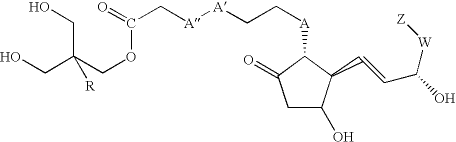

| Example | R | A | A′ | A″ | W | Z |
|---|---|---|---|---|---|---|
| 119 | Me | CH₂ | S | CH₂ | CH₂ | 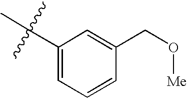 |
| 120 | Et | S | CH₂ | S | CH₂ | 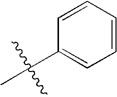 |
| 121 | H | S | CH₂ | S | CH₂ | 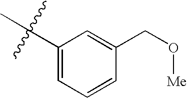 |
| 122 | CH₂OH | S | CH₂ | S | CH₂ | 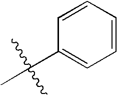 |
| 123 | Me | S | CH₂ | S | CH(CH₃) | 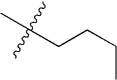 |
| 124 | OMe | CH₂ | S | CH₂ | CH₂O | 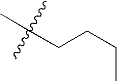 |

### Example 125

Preparation of (E125b)

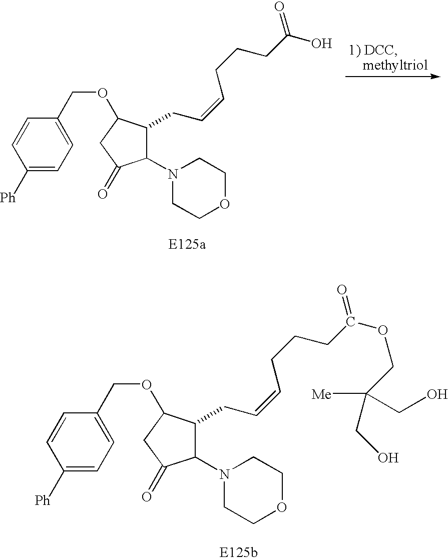

A mixture of [1α(Z),2β,5α]-7-{5-([1,1′-biphenyl]-4-yl methoxy)-2-(4-morphinyl)-3-oxocyclopentyl]-4-heptenoic acid (E86a) (available from Cayman Chemical Company, Ann Arbor, Mich.) and DCC in THF is allowed to stand at room temperature for 30 minutes and is then combined with the methyltriol, 2-(hydroxymethyl)-2-methylpropane-1,3-diol (15 equivalents). The reaction is allowed to stir for 24 hours at 25 degrees Centigrade. Ethyl Acetate/brine is used to partition the layers and the organic layer is and then the mixture is concentrated and purified by flash column chromatography to give the homotopic ester E125b.

### Examples 126-130

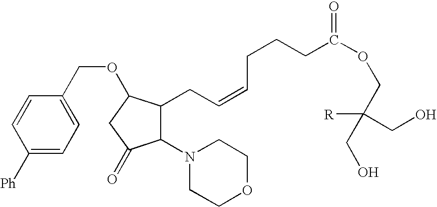

| Example | R |
|---|---|
| 126 | Me |
| 127 | CH₂OH |
| 128 | CH₂CH₃ |
| 129 | CH₂OCH₃ |
| 130 | Cl |

Examples 106-130 show that EP4 agonists can be synthesized according to this invention.

### Example 131

Preparation of 3-hydroxy-2-(hydroxymethyl)-2-methylpropyl butaprost (E131a):

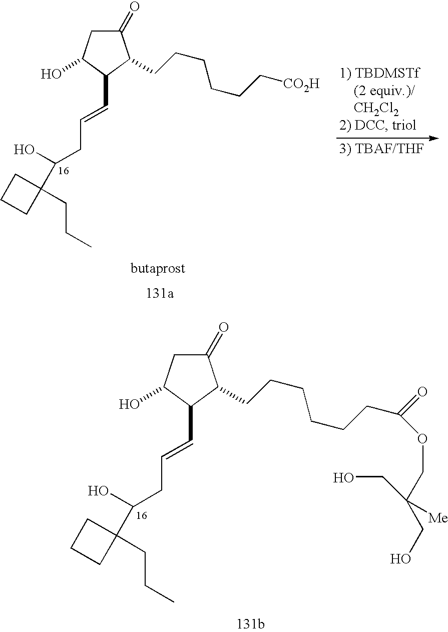

Butaprost free acid (Cayman Chemicals, Ann Arbor Mich.) is dissolved in methylene chloride and 2.3 equivalents of TBDMSTf and Lutidine (2.5 equiv.) are added. The solution is stirred at −78° C. while the TBDMSTf is added dropwise. The reaction is stirred for 1 hour and then allowed to warm to room temperature and stirred an additional 1 hour. The reaction mixture is quenched with water and the methylene chloride is removed under reduced pressure. The residue is dissolved in THF and DCC (1.05 equiv.) and 2-(hydroxymethyl)-2-methylpropane-1,3-diol (10 equiv.) are added. The reaction is allowed to stir overnight. The reaction is then washed with NH₄Cl₍ₛₐₜ₎, extracted with EtOAc, dried (Na₂SO₄), filtered, evaporated. Column chromatography (SiO₂, EtOAc/hexanes) gives 3-hydroxy-2-(hydroxymethyl)-2-methylpropyl butaprost as the bis-silyl adduct. This material is then treated with TBAF in THF at ambient temperature for 2 hours to effect removal of the silyl protecting groups. The mixture is concentrated and chromatography yields 3-hydroxy-2-(hydroxymethyl)-2-methylpropyl butaprost (E131b).

### Examples 132-156

Compounds 132-156 are made using the procedure set forth in Examples 131 and substituting the appropriate starting materials.

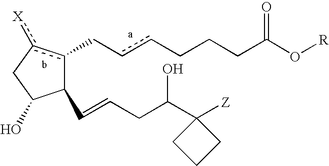

| R | a | b | X | Z |
|---|---|---|---|---|
| 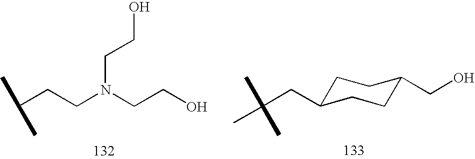 | CH₂CH₂ | C—CH | ═O | CH₂CH₂CH₃ |
| 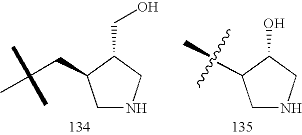 | | | | |
| 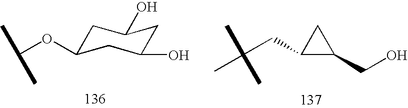 | CH═CH (cis) | C—CH | ═O | CH₂CH₂CH₃ |
| 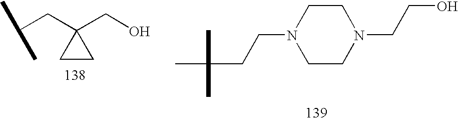 | | | | |
| 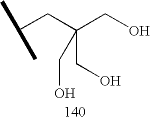 | | | | |
| 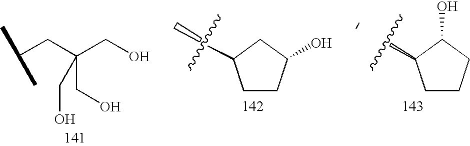 | CH═CH (cis) | C—CH | β-Cl | 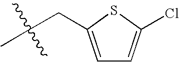 |
| 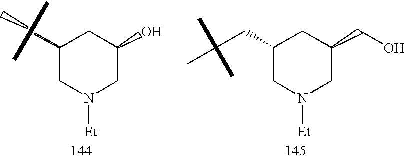 | | | | |
| 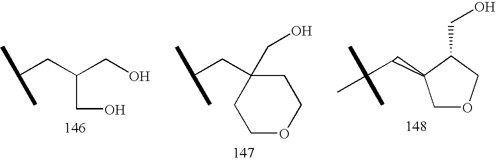 | CH═CH (cis) | C═C | F | 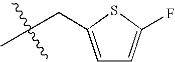 |
| 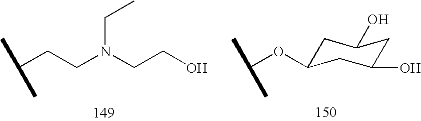 | | | | |
| 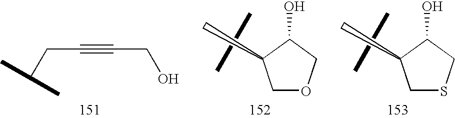 | CH═CH (cis) | C═C | H | 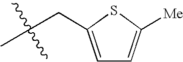 |
| 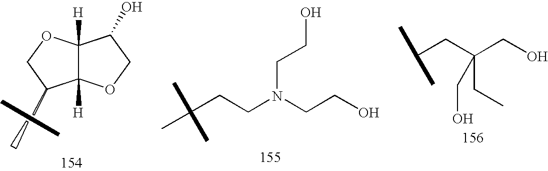 | | | | |

Examples 131-156 show that EP₂-selective ligands can be prepared according to this invention.

### Example 157

Preparation of 3-hydroxy-2,2-bis(hydroxymethyl)propyl 2-(2,3-dichloro-4-(thiophene-2-carbonyl)phenoxy)acetate (E157):

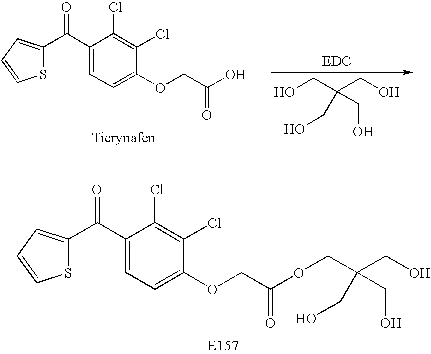

To ticrynafen in DMF is added EDC, DMAP and excess tetraol alcohol 2,2-bis(hydroxymethyl)propane-1,3-diol. This mixture is stirred overnight at 25° C. The reaction is washed with NH₄Cl₍ₛₐₜ₎, extracted with EtOAc, dried (Na₂SO₄), filtered, evaporated. Column chromatography (SiO₂, 5% MeOH/CH₂Cl₂) gives 3-hydroxy-2,2-bis(hydroxymethyl)propyl 2-(2,3-dichloro-4-(thiophene-2-carbonyl)phenoxy)acetate (E157).

### Example 158

Preparation of (E)-3-hydroxy-2,2-bis(hydroxymethyl)propyl 3-(4-(2-phenylacryloyl)phenyl)acrylate (E159):

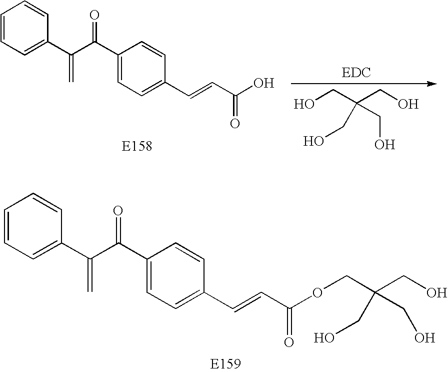

To cinnamic acid E158 in DMF is added EDC, DMAP and excess tetraol alcohol. This mixture is stirred overnight at 25° C. The reaction is washed with NH₄Cl₍ₛₐₜ₎, extracted with EtOAc, dried (Na₂SO₄), filtered, evaporated. Column chromatography (SiO₂, 5% MeOH/CH₂Cl₂) gives (E)-3-hydroxy-2,2-bis(hydroxymethyl)propyl 3-(4-(2-phenylacryloyl)phenyl)acrylate (E159).

### Examples 160-183

Using largely the procedure set forth in Examples 157 and 159 and substituting the appropriate starting materials, the compounds 160-183 are made.

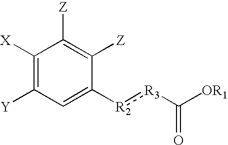

| R₁ | X | Y | Z | R₂═R₃ |
|---|---|---|---|---|
| 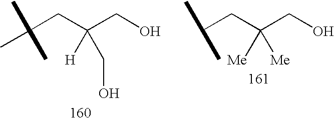 | 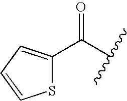 | H | Cl | O—CH₂ |
| 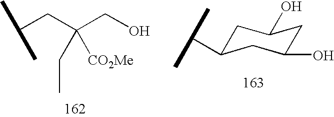 | 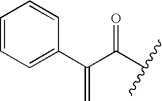 | H | H | CH═CH (trans) |
| 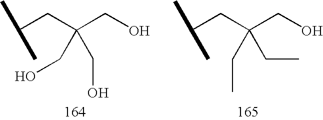 | 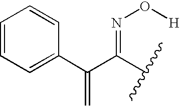 | H | H | O—CH₂ |
| 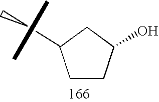 | | | | |
| 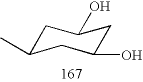 | 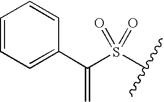 | H | H | CH═CH (trans) |
| 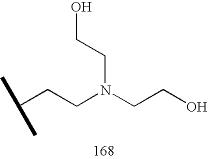 | | | | |
| 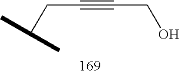 | | | | |
| 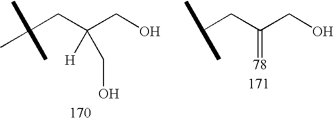 | 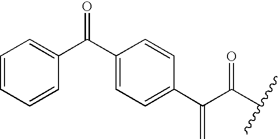 | H | H | CH═CH (trans) |
| 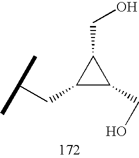 | | | | |
| 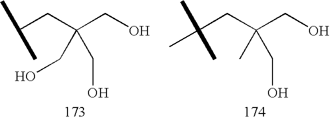 | 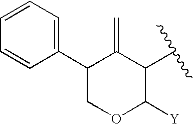 | 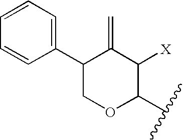 | H | CH═CH (trans) |
| 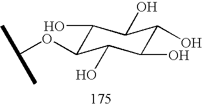 | | | | |
| 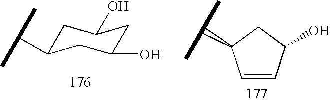 | 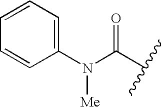 | H | H | CH═CH (trans) |
| 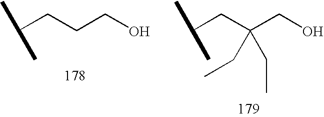 | | | | |
| 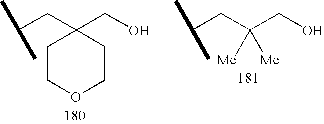 | 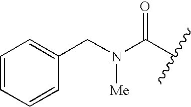 | H | H | CH═CH (trans) |
| 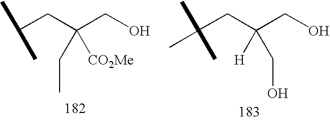 | | | | |

Examples 157-183 show that ethacrynic acid analogs can be prepared according to this invention.

### Example 184

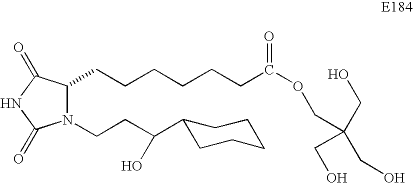

Prodrug of BW-245C

### Example 185

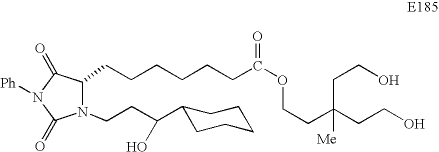

Prodrug of BW-868

Examples 184-185 show that DP-selective agonists and antagonists can be prepared according to this invention by the methods described above.

### Example 186

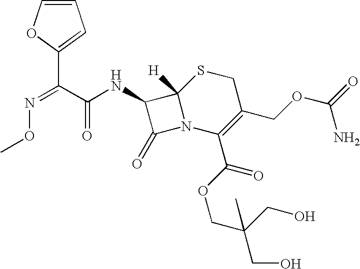

Prodrug of Cefuroxime

### Example 187

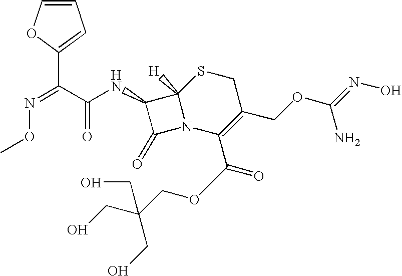

Prodrug of Ceftizoxime

Examples 186-187 show that derivatives of cephalosporin antibiotics can be prepared according to this invention using largely the methods shown above.

### Example 188

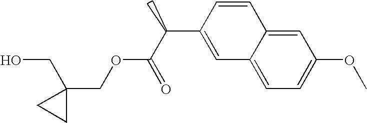

Prodrug of Naproxen

### Example 189

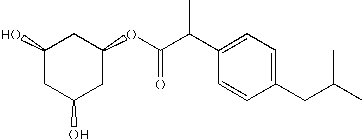

Prodrug of Ibuprofen

Examples 188-189 show that derivatives of non-steroidal anti-inflammatory agents can be prepared according to this invention using largely the methods shown above.

### Example 190

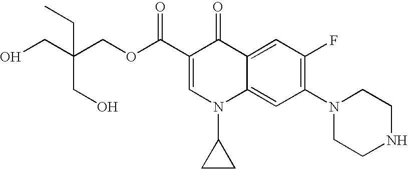

Prodrug of Ciprofloxacin

### Example 191

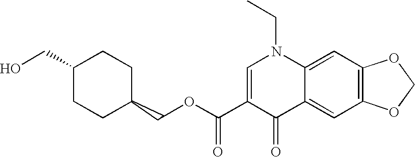

Prodrug of Oxolinic acid

Examples 190-191 show that derivatives of Quinolone antibiotics can be prepared according to this invention using largely the methods shown above.

### Example 192

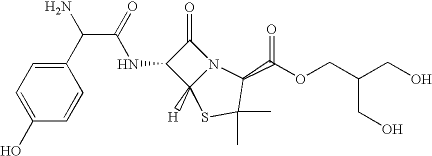

Prodrug of Amoxicillin

Example 192 shows that derivatives of Penicillin antibiotics can be prepared according to this invention using largely the methods shown above.

### Example 193

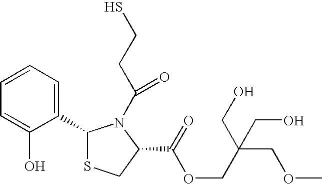

(2R,4R)-2-(2-Hydroxyphenyl)-3-(3-mercaptopropionyl)-4-thiazolidinecarboxylic acid

Example 193 shows that derivatives of angiotension-converting enzyme inhibitors can be prepared according to this invention using largely the methods shown above.

### Example 194

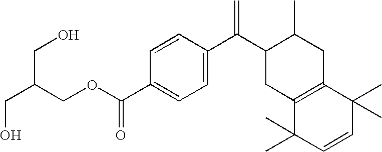

Prodrug of Bexarotene

### Example 195a

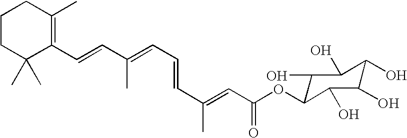

Prodrug of all-trans-retinoic acid

Examples 194-195a show that derivatives of Retinoids can be prepared according to this invention using largely the methods shown above.

### Example 195b

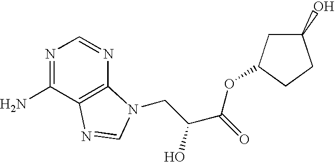

Prodrug of (R,S)-3-Adenin-9-yl-2-hydroxypropanoic acid

### Example 196

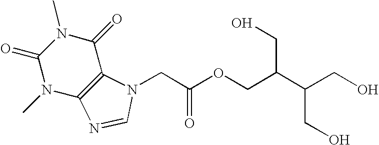

Prodrug of 7-Theophylline acetic acid

Examples 195b-196 show that other bio-affecting carboxylic acid agents can be prepared according to this invention using largely the methods shown above.

### Example 197

Pharmaceutical compositions in the form of tablets are prepared by conventional methods, such as mixing and direct compaction, formulated as follows:

| Ingredient | Quantity (mg per tablet) |
|---|---|
| Prostaglandin Derivative | 5 |
| Microcrystalline Cellulose | 100 |
| Sodium Starch Glycollate | 30 |
| Magnesium Stearate | 3 |

A derivative of an FP agonist according to this invention is used as the prostaglandin derivative. When administered orally once daily, the above composition substantially increases bone volume in a patient suffering from osteoporosis.

### Reference Example 1

### Pharmacological Activity for Glaucoma Assay

Pharmacological activity for glaucoma can be demonstrated using assays designed to test the ability of the subject compounds to decrease intraocular pressure. Examples of such assays are described in the following reference, incorporated herein: Liljebris, C.; Selen, G.; Resul, B.; Sternschantz, J.; Hacksell, U. “Derivatives of 17-Phenyl-18,19,20-trinorprostaglandin F_{2α} Isopropyl Ester: Potential Antiglaucoma Agents”, Journal of Medicinal Chemistry, Vol. 38 (2), 1995, pp. 289-304.

### Example 198

Pharmaceutical compositions in liquid form are prepared by conventional methods, formulated as follows:

| Ingredient | Quantity | |
|---|---|---|
| Prostaglandin Derivative | 1 | mg |
| Phosphate buffered physiological saline | 100 | ml |
| Methyl Paraben | 0.5 | mL |

A derivative of an EP₂ agonist according to this invention is used as the prostaglandin derivative. When 1.0 ml of the above composition is administered by ocular drops twice daily, the above composition substantially decreases intraocular pressure in a patient suffering from glaucoma.

### Example 199

Topical pharmaceutical compositions for lowering intraocular pressure are prepared by conventional methods and formulated as follows:

| Ingredient | Amount (wt %) |
|---|---|
| Prodrug Prostaglandin Derivative | 0.04 |
| Dextran 70 | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 7.0-7.2 |
| Purified water | q.s. to 100% |

A prodrug of an EP₂ agonist according to this invention is used as the prostaglandin derivative. When the composition is topically administered to the eyes once daily, the above composition decreases intraocular pressure in a patient suffering from glaucoma.

### Example 200

Example 199 is repeated using a prodrug of an EP₄ agonist according to this invention instead of the EP₂ agonist. When administered as a drop 4 times per day, the above composition substantially decreases intraocular pressure and serves as a neuroprotective agent.

### Example 201

Example 199 is repeated using a prodrug of an FP agonist according to this invention instead of the EP₂ agonist. When administered as a drop once per day, the above composition substantially decreases intraocular pressure.

### Example 202

Example 199 is repeated using a pro-DP antagonist according to this invention instead of the EP₂ agonist. When administered as a drop twice per day, the above composition substantially decreases allergic symptoms and relieves dry eye syndrome.

### Example 203

Example 199 is repeated using a pro-FP antagonist according to this invention instead of the EP₂ agonist. When administered as a drop as needed, the above composition substantially decreases hyperemia, redness and ocular irritation.

### Example 204

Example 199 is repeated using a pro-quinolone antibiotic according to this invention instead of the EP₂ agonist. When administered as a drop as needed, the above composition substantially reduces bacterial infections.

### Example 205

Compositions for topical administration are made, comprising:

Component

| | 204-1 | 204-2 | 204-3 | 204-4 |
|---|---|---|---|---|
| Pro-PGF agonist (wt %) | 0.01 | 0.1 | 1.0 | 10.0 |
| IC₅₀ the PGF (nM) | 1 | 10 | 100 | 1000 |
| Ethanol (wt %) | 59.99 | 59.9 | 59.4 | 54.0 |
| Propylene Glycol (wt %) | 20.00 | 20.0 | 19.8 | 18.0 |
| Dimethyl Isosorbide (wt %) | 20.00 | 20.0 | 19.8 | 18.0 |

A human male subject suffering from male pattern baldness is treated by a method of this invention. Specifically, for 6 weeks, one of the above compositions is twice daily administered topically to the subject.

### Example 206

Pharmaceutical compositions in liquid form are prepared by conventional methods, formulated as follows:

| Ingredient | Quantity | |
|---|---|---|
| Prodrug Prostaglandin Derivative | 100 | mg |
| Phosphate buffered physiological saline | 10 | mL |
| Methyl Paraben | 0.05 | mL |

An pro-FP antagonist according to this invention is used as the prostaglandin derivative. When 0.25 mL/min of the above composition is instilled into a uterus undergoing premature labor, the above composition decreases uterine contractions within the first hour, preventing premature birth.

### Example 207

### Testing the Release Rate of the Symmetrical Alcohol Esters Using Purified Butyrylcholinesterase

Commercially available butyrylcholinesterase (CAS#9001-08-5) is prepared as an aqueous solution containing 5 mg of (NH₄)₂SO₄ per mg of protein. One unit of butyrylcholinesterase corresponds to the amount of enzyme which hydrolyzes 1 micromole of butyrylcholine per minute at pH 8.0 and 25° C. The symmetrical alcohol esters, from Example X above, are dissolved in ethanol at various concentrations and added to the assay such that the final ethanol concentration equals 10%. The remaining 90% of the reaction mix consists of appropriate and commonly used buffer components. At 15, 30, 60, 90, 120, 240 and 360 minutes, aliquots from the reaction are removed and enzymatic activity terminated by addition of excess acetonitrile. Hydrolysis of the esters is monitored by LC/MS with UV detection using pure esters and corresponding acids as reference standards. A relative rate, at each concentration of symmetrical alcohol, is determined for each ester which allows: 1) the ranking of said esters according to their susceptibility to hydrolysis by butyrylcholinesterase and, 2) the concentration of compound required to release a pre-defined amount of carboxylic acid within a pre-defined time period.

### Example 208

### Testing the Release Rate of the Symmetrical Alcohol Esters Using Purified Human Corneal Epithelial Cell Homogenate

Commercially available immortalized human corneal epithelial cells are cultured according to recommended methods. The corneal epithelial cell homogenate is prepared by disruption of the cells in a buffered solution containing protease inhibitors and appropriate protein stabilizing agents. The homogenate is used immediately or frozen until the time of use. The symmetrical alcohol esters, from Example X above, are dissolved in ethanol and added to the assay such that the final ethanol concentration equals 10%. The remaining 90% of the reaction mix consists of appropriate and commonly used buffer components. At 15, 30, 60, 90, 120, 240 and 360 minutes, aliquots from the reaction are removed and enzymatic activity terminated by addition of excess acetonitrile. Hydrolysis of the esters is monitored by LC/MS with UV detection using pure esters and corresponding acids as reference standards. A relative rate at each concentration of symmetrical alcohol is determined for each ester which allows: 1) the ranking of said esters according to their susceptibility to hydrolysis by enzymes present in the human corneal epithelium and, 2) the concentration of compound required to release a pre-defined amount of carboxylic acid within a pre-defined time period.

That which is claimed is:

1. A compound having the formula: 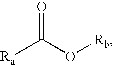
wherein 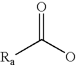
is a biologically-active moiety comprising a carboxylic acid functional group; and
R_{b} is a homotopically-symmetrical alcohol bonded to the biologically-active moiety through the carboxylic acid functional group to form an ester linkage, as well as
optical isomers, enantiomers, pharmaceutically acceptable salts, biohydrolyzable amides, esters, and imides thereof and combinations thereof.

2. The compound of claim 1, wherein the homotopically-symmetrical alcohol has the formula 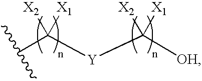
wherein X₁ and X₂ are independently selected from the group consisting of H, a halogen atom, a hydroxymethyl group, a lower monovalent hydrocarbon group, a lower heterogeneous group, an alkoxymethyl group, an aryloxymethyl group, an amino group, a heterogeneous group, a carbocyclic group, a heterocyclic group, an aromatic group, a heteroaromatic group, a substituted carbocyclic group, a substituted heterocyclic group, a substituted aromatic group, and a substituted heteroaromatic group;
Y is a bond, which may be single, double or triple, or is selected from the group consisting of (CX₁X₂)ₘ, O, NX₁, S, SO₂, and alternating or repeating units thereof; and
m and n are independently integers from zero to nine.

3. The compound of claim 2, wherein the homotopically-symmetrical alcohol has the formula 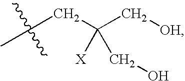
wherein X is selected from the group consisting of H, a halogen atom, a hydroxymethyl group, a lower monovalent hydrocarbon group, a lower heterogeneous group, an alkoxymethyl group, an aryloxymethyl group, an amino group, a heterogeneous group, a carbocyclic group, a heterocyclic group, an aromatic group, a heteroaromatic group, a substituted carbocyclic group, a substituted heterocyclic group, a substituted aromatic group, and a substituted heteroaromatic group.

4. The compound of claim 2, wherein the homotopically-symmetrical alcohol has the formula

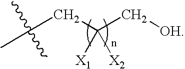

5. The compound of claim 2, wherein the homotopically-symmetrical alcohol is a triol.

6. The compound of claim 2, wherein the homotopically-symmetrical alcohol is a tetraol.

7. The compound of claim 1, wherein the homotopically-symmetrical alcohol has the formula 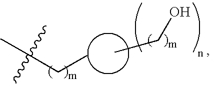
wherein the circle represents an alicyclic ring of from about 3 to about 7 member atoms;
m is an integer of from about zero to about 4 member atoms; and
n is an integer of from about one to about 7 groups, whereby the groups are located symmetrically about the alicyclic ring.

8. The compound of claim 1, wherein the biologically-active moiety is a prostaglandin.

9. The compound of claim 1, wherein the biologically-active moiety is at least one of travaprost, latanoprost, bimatoprost and combinations thereof.

10. The compound of claim 1, wherein the biologically-active moiety has the formula 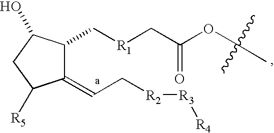
wherein R¹ is a divalent group selected from the group consisting of a hydrocarbon group, a substituted hydrocarbon group, a heterogeneous group, and a substituted heterogeneous group, with the proviso that when R¹ is a heterogeneous group, R¹ has only one heteroatom selected from the group consisting of oxygen, sulfur, and nitrogen;
R² is a divalent group selected from the group consisting of —C(O)— and —C(R⁶)(OR⁷)— and —CF₂—;
R³ is a divalent group having the formula —(CD(D))ₚ-X—(CD(D))_{q}-, wherein p is an integer from 0 to 3 and q is an integer from 0 to 3, X is selected from the group consisting of an oxygen atom, a divalent hydrocarbon group, a sulfur atom, SO, SO₂, and ND, and D is selected from the group consisting of a hydrogen atom, a monovalent hydrocarbon group of 1 to 4 carbon atoms, and a monovalent heterogeneous group of 1 to 4 member atoms;
R⁴ is selected from the group consisting of a methyl group, a carbocyclic group, a substituted carbocyclic group, a heterocyclic group, a substituted heterocyclic group, an aromatic group, a substituted aromatic group, a heteroaromatic group, and a substituted heteroaromatic group;
R⁵ is selected from the group consisting of a hydrogen atom, a halogen atom, NHR⁶, a carbonyl group and OR⁶;
R⁶ and R⁷ are independently selected from the group consisting of a hydrogen atom, a monovalent hydrocarbon group of 1 to 4 carbon atoms, and a monovalent heterogeneous group of 1 to 4 member atoms; and
bond a is selected from the group consisting of a triple bond, a single bond and a double bond.

11. The compound of claim 1, wherein the biologically-active moiety has the formula 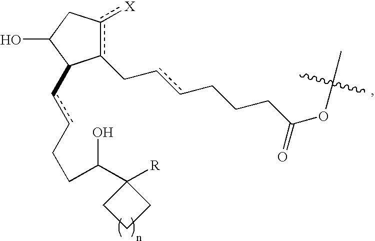
wherein X is H, OH, a halogen or a carbonyl;
R is a lower monovalent hydrocarbon group, a lower heterogeneous group, an aromatic group or a heteroaromatic group, each of which may be substituted or unsubstituted;
n is an integer from 0 to about 4; and
the solid and dashed lines together indicate a single or double bond.

12. The compound of claim 1, wherein the biologically-active moiety has the formula 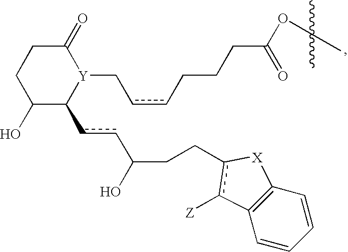
wherein X is selected from NH, S, CH₂, C═O, O, or SO₂;
Y is N or CH;
Z is a halogen, a lower monovalent hydrocarbon group or a lower heterogeneous group; and
the solid and dashed lines together indicate single or double bonds.

13. The compound of claim 1, wherein the biologically-active moiety has the formula 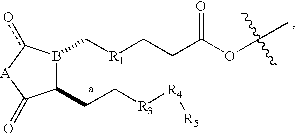
wherein R¹ is a divalent group selected from the group consisting of a hydrocarbon group, a substituted hydrocarbon group, a heterogeneous group, and a substituted heterogeneous group, with the proviso that when R¹ is a heterogeneous group, R¹ has only one heteroatom selected from the group consisting of oxygen, sulfur, and nitrogen;
R² is a divalent group selected from the group consisting of —C(O)— and —C(R⁶)(OR⁷)— and —CF₂—;
R³ is a divalent group having the formula —(CD(D))ₚ-X—(CD(D))_{q}-, wherein p is an integer from 0 to 3 and q is an integer from 0 to 3, X is selected from the group consisting of an oxygen atom, a divalent hydrocarbon group, a sulfur atom, SO, SO₂, and ND, and D is selected from the group consisting of a hydrogen atom, a monovalent hydrocarbon group of 1 to 4 carbon atoms, and a monovalent heterogeneous group of 1 to 4 member atoms;
R⁴ is selected from the group consisting of a methyl group, a carbocyclic group, a substituted carbocyclic group, a heterocyclic group, a substituted heterocyclic group, an aromatic group, a substituted aromatic group, a heteroaromatic group, and a substituted heteroaromatic group;
R⁵ is selected from the group consisting of a hydrogen atom, a halogen atom, NHR⁶, a carbonyl group and OR⁶;
R⁶ and R⁷ are independently selected from the group consisting of a hydrogen atom, a monovalent hydrocarbon group of 1 to 4 carbon atoms, and a monovalent heterogeneous group of 1 to 4 member atoms;
A is selected from the group consisting of CH₂ and NR⁶;
B is selected from the group consisting of —CH and N; and
bond a is selected from the group consisting of a triple bond, a single bond and a double bond.

14. The compound of claim 1, wherein the biologically-active moiety has the formula 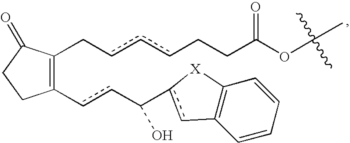
wherein X is selected from the group consisting of CH₂, NR, S, O and SO₂;
R is selected from H, a methyl group, a hydrocarbon group, a substituted hydrocarbon group, a heterogeneous group, and a substituted heterogeneous group, a carbocyclic group, a substituted carbocyclic group, a heterocyclic group, a substituted heterocyclic group, an aromatic group, a substituted aromatic group, a heteroaromatic group, and a substituted heteroaromatic group; and
the solid and dashed lines together indicate single or double bonds.

15. The compound of claim 1, wherein the biologically-active moiety has the formula 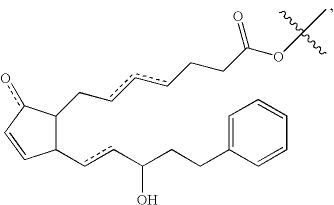
wherein the solid and dashed lines together indicate single or double bonds.

16. The compound of claim 1, wherein the biologically-active moiety is ethacrynic acid.

17. The compound of claim 1, wherein the biologically-active moiety has the formula 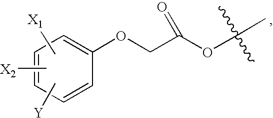
wherein X₁ and X₂ are independently selected from the group consisting of H, halogen, NR₁R₂, SR₁ and OR₁;
R₁ and R₂ are each independently H, a lower monovalent hydrocarbon group or a lower heterogeneous group; and
Y is a substituted or unsubstituted lower monovalent hydrocarbon group, lower heterogeneous group, aromatic group or heteroaromatic group.

18. The compound of claim 1, wherein the biologically-active moiety has the formula 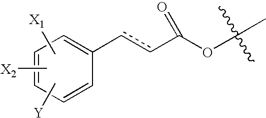
wherein X₁ and X₂ are independently selected from the group consisting of H, halogen, NR₁R₂, SR₁ and OR₁;
R₁ and R₂ are each independently H, a lower monovalent hydrocarbon group or a lower heterogeneous group;
Y is a substituted or unsubstituted lower monovalent hydrocarbon group, lower heterogeneous group, aromatic group or heteroaromatic group; and
the solid and dashed lines together indicate a single or double bond.

19. The compound of claim 1, having the formula 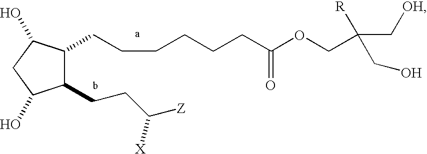
wherein R is selected from the group consisting of H, methyl, ethyl and CH₂OH;
a and b are independently selected from the group consisting of CH═CH and CH₂CH₂;
X is selected from the group consisting of OH and O; and
Z is selected from the group consisting of

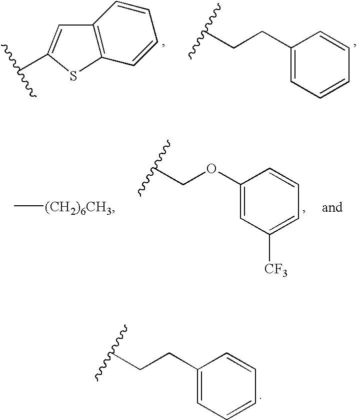

20. The compound of claim 19, wherein R is selected from the group consisting of H, methyl and ethyl;
a and b are each CH₂CH₂;
X is OH; and
Z is

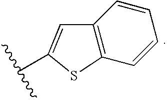

21. The compound of claim 19, wherein R is selected from the group consisting of H, methyl, ethyl and CH₂OH;
a is CH═CH;
b is CH₂CH₂;
X is OH; and
Z is

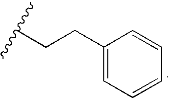

22. The compound of claim 19, wherein R is selected from the group consisting of H, methyl, ethyl and CH₂OH;
a is CH═CH;
b is CH₂CH₂;
X is O; and
Z is —(CH₂)₆CH₃.

23. The compound of claim 19, wherein R is selected from the group consisting of H, methyl, ethyl, CH₂OH;
a and b are CH═CH;
X is OH; and
Z is

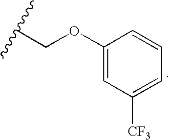

24. The compound of claim 19, wherein R is selected from the group consisting of H, methyl, ethyl, and CH₂OH;
a and b are CH═CH;
X is OH; and
Z is

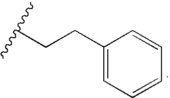

25. A composition comprising:
A) a compound having the formula 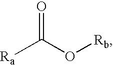
wherein 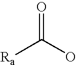
is a biologically-active moiety comprising a carboxylic acid functional group; and
R_{b} is a homotopically-symmetrical alcohol bonded to the biologically-active moiety through the carboxylic acid functional group to form an ester linkage, as well as
optical isomers, enantiomers, pharmaceutically acceptable salts, biohydrolyzable amides, esters, and imides thereof and combinations thereof.
B) a carrier.

26. The composition of claim 25, wherein the compound has the formula 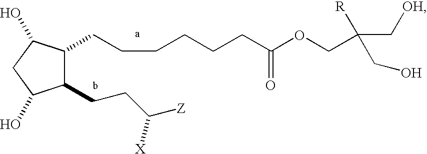
wherein R is selected from the group consisting of H, methyl, ethyl and CH₂OH;
a and b are independently selected from the group consisting of CH═CH and CH₂CH₂;
X is selected from the group consisting of OH and O; and
Z is selected from the group consisting of

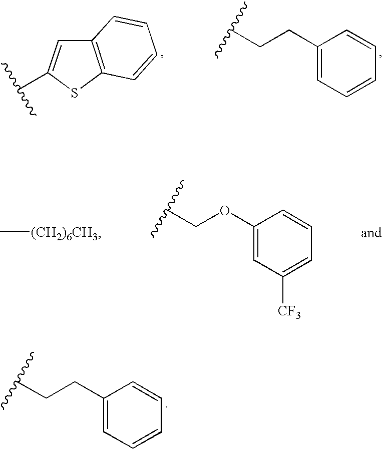

27. The composition of claim 25, wherein the carrier is selected from the group consisting of systemic and topical carriers.

28. The composition of claim 25, wherein the carrier is a systemic carrier that comprises one or more ingredients selected from the group consisting of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, and combinations thereof.

29. The composition of claim 28, wherein the composition comprises about 0.01% to 50% of the compound and 50 to 99.99% of the systemic carrier.

30. The composition of claim 25, wherein the carrier is a topical carrier that comprises one or more ingredients selected from the group consisting of water, alcohols, aloe vera gel, allantoin, glycerin, vitamin A and E oils, mineral oil, propylene glycol, PPG-2 myristyl propionate, dimethyl isosorbide, q) emollients, r) propellants, s) solvents, t) humectants, u) thickeners, v) powders, w) fragrances, x) pigments, y) sugars, z) cellulose or a derivative thereof, aa) a salt, bb) Edetate disodium, cc) a pH adjusting additive, and combinations thereof.

31. The composition of claim 30, wherein the composition comprises 0.0001% to about 1% of the compound.

32. A method for treating a condition comprising administering to a subject in need of treatment, a homotopic prodrug of a prostaglandin EP₂ agonist, wherein the condition is selected from the group consisting of glaucoma, ocular hypertension, premenstrual tension, asthma and bone disorders.

33. The method of claim 32, wherein the condition is selected from the group consisting of glaucoma and bone disorders.

34. A method for treating a condition comprising administering to a subject in need of treatment, a homotopic prodrug of a prostaglandin EP₃ agonist, wherein the condition is selected from the group consisting of arthritis, bone disorders, vascular disease, hepatic diseases, renal diseases, pancreatitis, mycardial infarct, and gastric disturbances.

35. The method of claim 34, wherein the condition is bone disorders.

36. A method for controlling blood pressure comprising administering to a subject in need of treatment, a homotopic prodrug of an angiotensin-converting enzyme inhibitor.

37. A method for treating a condition comprising administering to a subject in need of treatment, a homotopic prodrug of a prostaglandin EP₄ agonist, wherein the condition is selected from the group consisting of glaucoma, ocular hypertension, neuroprotection, arthritis, bone disorders, vascular disease, and asthma.

38. The method of claim 37, wherein the condition is selected from the group consisting of glaucoma and bone disorders.

39. A method for treating a condition comprising administering to a subject in need of treatment, a homotopic prodrug of a prostaglandin FP agonist, wherein the condition is selected from the group consisting of glaucoma, skin disorders, circulatory disorders, gastrointestinal disorders, vascular diseases, and respiratory disorders.

40. The method of claim 39, wherein the condition is glaucoma.

41. A method for preventing premature labor comprising administering to a subject in need of treatment, a homotopic prodrug of a prostaglandin FP antagonist.

42. A method for treating sleeping disorders comprising administering to a subject in need of treatment, a homotopic prodrug of a prostaglandin DP agonist.

43. A method for treating allergies comprising administering to a subject in need of treatment, a homotopic prodrug of a prostaglandin DP antagonist.

44. A method of ranking the susceptibility of an ester derivative to hydrolysis, said method comprising:
producing an ester derivative by reacting a biologically-active moiety having a carboxylic acid group with a homotopically-symmetrical alcohol that binds the biologically-active moiety through the carboxylic acid group to form an ester linkage;
reacting the ester derivative with an enzyme that cleaves the ester linkage and releases the biologically-active moiety;
measuring the rate at which the enzyme cleaves the ester linkage; and
ranking the ester derivative for therapeutic usefulness.

45. The method of claim 44, wherein butyrylcholinesterase is used in the determining step.

46. The method of claim 44, wherein the biologically-active moiety comprises a phenoxyacetic acid

47. The method of claim 44, wherein the biologically-active moiety comprises a cinnamic acid.

48. The method of claim 44, wherein the biologically-active moiety comprises a prostaglandin.

49. The method of claim 44, wherein the predetermined half-life is greater than about one minute but less than about seven days.

50. The method of claim 44, wherein the predetermined half-life is greater than about five minutes and less than about 24 hours.

51. A method of treating an ophthalmologic disorder comprising the steps of claim 44 and further comprising administering to a human or other animal a safe and effective amount of the compound.
